(19) United States
(12) Patent Application Publication
(10) Pub. No.: US 2019/0374572 A1
(43) Pub. Date: Dec. 12, 2019

(54) KAOLINIC CLAYS WITH ANTIMICROBIAL ACTIVITY

(71) Applicant: IMERYS USA, INC., Roswell, GA (US)

(72) Inventors: Stacey JOHNSON, Cochran, GA (US); Sarah JOHNSON, Brunswick, GA (US); Anthony LYONS, Macon, GA (US); Chris BOOTHBY, Cumming, GA (US); J. Phillip E. JONES, Woodstock, GA (US)

(21) Appl. No.: 16/483,310

(22) PCT Filed: Feb. 1, 2018
(86) PCT No.: PCT/US2018/016464

§ 371 Date: Aug. 2, 2019

Related U.S. Application Data
(60) Provisional application No. 62/454,691, filed on Feb. 3, 2017.

(51) Int. Cl. A61K 33/06; A61K 33/26; A61K 33/34; A61K 9/00; A61K 9/10

(52) U.S. Cl. CPC A61K 33/06; A61K 33/26; A61K 9/10; A61K 9/0014; A61K 33/34

(57) ABSTRACT

This disclosure describes antimicrobial compositions containing a clay, an aluminum compound and optionally a transition metal compound, in which a pH of the antimicrobial composition is less than or equal to 5, and an oxidation-reduction potential (ORP) of the antimicrobial composition ranges from about 300 mV to about 800 mV. Other embodiments described herein include methods of producing and using the antimicrobial compositions.

### CLAIM FOR PRIORITY

This PCT International Application claims the benefit of priority of U.S. Provisional Patent Application No. 62/454,691, filed Feb. 3, 2017, the subject matter of which is incorporated herein by reference in its entirety.

### TECHNICAL FIELD

This application relates to materials technology in general and more specifically to the preparation and use of antimicrobial compositions. More particularly, this application discloses the preparation and use of antimicrobial compositions containing a clay or clay-like compound, such as a kaolinic clay, and an aluminum compound. Antimicrobial compositions disclosed herein are useful for treating infections and diseases caused by bacteria and other microbes, and are also useful for treating and protecting living or non-living surfaces against microbial activity.

### BACKGROUND

Antibiotics and similar drugs, together called antimicrobial agents, have been used for the last 70 years to treat patients who have infectious diseases. Although antimicrobial drugs have greatly reduced illness and death from infectious diseases, the widespread use and misuse of these agents has allowed infectious organisms to adapt and mutate into drug-resistant forms for which known antimicrobial drugs are less effective.

One strategy for addressing the emergence of drug-resistant forms of antimicrobial agents is to employ mixtures of antimicrobial agents that can act to inhibit or kill the microbes via different mechanisms. For example, studies have been conducted exploring the potential use of clay minerals in the topical treatment of bacterial infections. These studies have shown that some natural and synthetic clays can be effectively used as antimicrobial agents. It is also recognized that some natural and synthetic clays can exhibit antimicrobial activity against drug-resistant forms of pathogens such as drug-resistant bacteria.

A clay is a fine-grained natural rock or soil material that combines one or more clay minerals with traces of metal oxides and organic matter. Clay materials develop plasticity when mixed with water, which is one reason why clays are believed to exhibit antimicrobial activity when used to topically treat infectious diseases. Due to their plasticity, the form of clay materials is well suited for topical treatments such as bandages and wound dressings. The air impermeability of clays is believed to enhance their antimicrobial activity by excluding oxygen, while the chemical content of certain clays is known to include some antimicrobial compounds that can further enhance antimicrobial activity.

Although it is currently known that the antimicrobial activity of some clays can be enhanced by adding reducing agents such as polymorphs of FeS₂, the development of clays as effective antimicrobial agents is still at an early stage.

### SUMMARY

The present inventors have recognized that a need exists to discover antimicrobial clay compositions exhibiting enhanced antimicrobial activity compared to natural clay materials. For example, a need exists for antimicrobial clay compositions that not only inhibit the growth of microbes such as bacteria, but also can partially or completely kill microbes. A need also exists for antimicrobial clay composition capable of performing these functions during treatment of infected subjects, and for performing these functions as disinfectants applied to surfaces in order to reduce or prevent infections. A need also exists for antimicrobial clay compositions that exhibit antimicrobial activity against drug-resistant strains of microbes such as bacteria, viruses, protozoa and fungi that are currently difficult to treat using traditional antibiotics.

Embodiments of the present disclosure, described herein such that one of ordinary skill in this art can make and use them, include the following:

(1) Some embodiments relate to an antimicrobial composition, comprising a clay and an aluminum compound, wherein a pH of the antimicrobial composition is less than or equal to 5, and an oxidation-reduction potential (ORP) of the antimicrobial composition ranges from about 300 mV to about 800 mV;

(2) Some embodiments relate to the antimicrobial composition (1), wherein the clay comprises a transition metal compound-such as, for example, wherein a crystalline structure of the clay includes the transition metal compound;

(3) Some embodiments relate to the antimicrobial composition (1), further comprising a transition metal compound as a separate component from the clay and the aluminum compound;

(4) Some embodiments relate to a method for producing the antimicrobial composition (1), comprising combining the clay and the aluminum compound to obtain the antimicrobial composition;

(5) Some embodiments relate to a method for producing the antimicrobial composition (3), comprising combining the clay, the aluminum compound and the transition metal compound to obtain the antimicrobial composition;

(6) Some embodiments relate to a method for reducing bacterial viability of one or more bacteria, comprising applying a bactericidal effective amount of the antimicrobial composition (1) or (3) to the one or more bacteria; and

(7) Some embodiments relate to a method for treating or preventing a bacterial infection in or on a subject, in which the bacterial infection is caused by one or more bacteria selected from the group consisting of E. coli, ESBL E. coli, M. marinum, Mycobacterium ulcerans, MRSA, M. smegmatis, Pseudomonas aeruginosa, Salmonella typhimurium, Staphylococcus epidermidis, S. aureus, and Streptococcus sp, the method comprising administering a bactericidal effective amount of the antimicrobial composition (1) or (3) to a subject in need thereof at a site of the bacterial infection.

Additional objects, advantages and other features of the present disclosure will be set forth in part in the description that follows and in part will become apparent to those having ordinary skill in the art upon examination of the following or may be learned from the practice of the present disclosure. The present disclosure encompasses other and different embodiments from those specifically described below, and the details herein are capable of modifications in various respects without departing from the present invention. In this regard, the description herein is to be understood as illustrative in nature, and not as restrictive.

### BRIEF DESCRIPTION OF THE DRAWINGS

Embodiments of this disclosure are explained in the following description in view of figures that show:

FIG. 1 is a graph showing the logarithmic relationship between the colony forming unit (CFU) concentration versus time for an E. coli culture;

FIG. 2 is a graph showing the relationship between the optical density of UV visible light at 570 nm versus time for an E. coli culture;

FIG. 3 is a graph showing the correlation of optical density (OD) to colony forming unit (CFU) concentration for an E. coli culture at 18 hours; and

FIG. 4 is a chart of antibacterial clay (ABC) potential versus optical density (OD) at 570 nm.

### DETAILED DESCRIPTION

Embodiments of this disclosure include various antimicrobial compositions, as well as processes for producing antimicrobial compositions, and methods of using antimicrobial compositions for treating infections and protecting surfaces against various microbes. The terms “about” and “approximately” as used herein refer to being nearly the same as a referenced amount or value, and should be understood to encompass ±5% of the specified amount or value.

### Antimicrobial Compositions

Some embodiments relate to antimicrobial compositions containing a clay and an aluminum compound. Such antimicrobial compositions may exist in the form of a solid such as a powder, or in the form of a clay-like material exhibiting physical plasticity in dry or moist form, or in the form of an aqueous or non-aqueous dispersion, or in the form of an aqueous or non-aqueous solution, just to name a few.

The term “antimicrobial” refers to an agent that kills microorganisms or inhibits their growth. Antimicrobial compositions of the present disclosure may act as “antibiotics” that kill or inhibit bacteria, as “antifungals” that kill or inhibit fungi, as “antivirals” that kill or inhibit viruses, and as “antiparasitics” that kill or inhibit parasites, just to name a few. Antimicrobial compositions of the present disclosure may also act as “disinfectants” that kill or inhibit a wide range of microbes on non-living surfaces to inhibit or prevent the spread of illness, and may also act as “antiseptics” that kill or inhibit a wide variety of microbes on living tissue to inhibit or prevent infections.

The aluminum compound may be a compound added to the clay, or the aluminum compound may be the product of a compound or material added to the clay. For example, in some embodiments the aluminum compound is provided by adding aluminum metal or an aluminum alloy to the clay, and the aluminum metal or aluminum alloy is converted into the aluminum compound.

In some embodiments the aluminum compound may be selected from aluminum, an alloy containing aluminum, aluminum acetate [Al(C₂H₃O₂)₃], aluminum sulfate [Al₂(SO₄)₃], potassium aluminum sulfate [KAl(SO₄)₂], aluminum carbonate [Al₂(CO₃)₃], aluminum sulfite [Al₂(SO₃)₃], aluminum oxide [Al₂O₃], aluminum chlorate [Al(ClO₃)₃], aluminum sulfide [Al₂S₃], aluminum nitrate [Al(NO₃)₃], aluminum permanganate [Al(MnO₄)₃], aluminum hydrogen carbonate [Al(HCO₃)₃], aluminum phosphate [AlPO₄], aluminum oxalate [Al₂(C₂O₄)₃], aluminum hydrogen phosphate [Al₂(HPO₄)₃], aluminum thiosulfate [Al₂(S₂O₃)₃], aluminum chlorite [Al(ClO₂)₃], aluminum hydrogen sulfate [Al(HSO₄)₃], aluminum dihydrogen phosphate [Al(H₂PO₄)₃], aluminum phosphite [AlPO₃], aluminum potassium sulfate [KAl(SO₄)₂], hydrates thereof, and mixtures thereof, just to name a few.

In some embodiments a d₅₀ of the aluminum compound may range from about 1 nm to about 1000 nm. For example, a d₅₀ of the aluminum compound may range from about 100 nm to about 900 nm, or from about 200 nm to about 800 nm, or from about 300 nm to about 700 nm, or from about 400 nm to about 600 nm. As explained below in greater detail, in some embodiments the antimicrobial composition also contains a transition metal compound. In such embodiments a d₅₀ of the transition metal compound may range from 1 nm to 1000 nm. For example, a d₅₀ of the transition metal compound may range from about 100 nm to about 900 nm, or from about 200 nm to about 800 nm, or from about 300 nm to about 700 nm, or from about 400 nm to about 600 nm.

In some embodiments a pH of the antimicrobial composition may be less than or equal to about 5. For example, a pH of the antimicrobial composition may range from about 2 to about 5, or from about 3 to about 5, or from about 2 to about 4, or from about 3.5 to about 4.5, or from about 4 to about 5. The pH of an antimicrobial composition of the present disclosure is measured by preparing an aqueous dispersion of the antimicrobial composition and then measuring pH using the procedure described in the experimental section below. In some embodiments it is observed that lowering the pH of the antimicrobial composition to a value of less than or equal to about 5 increases its antimicrobial activity. However, in some embodiments it is observed that lowering the pH of the antimicrobial composition to a value of less than about 2 can be detrimental to a living subject to be treated, or a surface to be disinfected, due to oxidative reactivity and toxicity below a pH of about 2.

In some embodiments an oxidation-reduction potential (ORP) of the antimicrobial composition may range from about 300 mV to about 800 mV. For example, an ORP of the antimicrobial composition may range from about 350 mV to about 500 mV, or from about 400 mV to about 600 mV, or from about 450 mV to about 550 mV. The ORP of an antimicrobial composition is measured by preparing an aqueous dispersion of the antimicrobial composition and then measuring ORP using the procedure described in the experimental section below. In some embodiments it was observed that adjusting the ORP of the antimicrobial composition to range from about 300 mV to about 800 mV increases its antimicrobial activity. For example, some antibacterial compositions containing the clay and the aluminum compound are especially potent against E. coli when the ORP is adjusted to range from about 400 mV to about 700 mV.

In some embodiments the particle size distribution of the clay can affect the antimicrobial activity of the antimicrobial composition. For example, in some embodiments the clay is a clay having a particle size distribution such that greater than about 20% by weight and less than about 60% by weight of particles of the fine clay have a particle size of less than 0.25 microns as measured by Sedigraph. In other embodiments the proportion of particles having a particle size of less than 0.25 microns can range from about 25% by weight to about 60% by weight, or from about 35% by weight to about 55% by weight, or from about 40% by weight to about 50% by weight.

Antimicrobial compositions of the present disclosure may include any natural or synthetic clay or clay material known in the relevant art. In some embodiments the clay is selected from a bentonite clay, a chlorite clay, an illite clay, a kaolinic clay, a montmorillonite clay, a rectorite clay, a smectite clay, or a mixture thereof, just to name a few. In some embodiments the clay may be a “kaolinic clay,” which means a clay or clay-like material containing a chemical composition having the formula Al₂Si₂O₅(OH)₄. In some embodiments the clay is a kaolinic clay such as kaolin. Kaolinic clays can be especially effective as clays used in antimicrobial compositions of the present disclosure. Embodiments of the present disclosure may include antimicrobial compositions containing at least two different clays or clay-like materials. Different clays may include different types of clays or different sub-types of a certain clay.

In some embodiments the antimicrobial composition may include a clay containing: 0.5-5.0 wt % of Fe₂O₃; 0.0-1.0 wt % of MgO; 10.0-50.0 wt % of Al₂O₃; 10.0-50.0 wt % of SiO₂; 1.0-5.0 wt % of TiO₂; 0.1-1.0 wt % of CaO; 0.1-2.0 wt % of Na₂O; 0.1-1.0 wt % of K₂O; 0.05-1.0 wt % of P₂O₅; 0.0-5.0 wt % of Horiba S; and 0.01-7.0 wt % of FeS₂, relative to a total weight of the clay.

In some embodiments the clay of the antimicrobial composition has a BET surface area of at least about 25 m²/g. For example, in some embodiments the clay has a BET surface area of at least about 30 m²/g, or at least about 40 m²/g. In other embodiments the clay is a kaolinic clay having a BET surface area ranging from about 25 m²/g to about 40 m²/g. BET surface area, as used herein, refers to the technique for calculating specific surface area of physical absorption molecules according to Brunauer, Emmett, and Teller (“BET”) theory. BET surface area may be measured by any appropriate measurement technique now known to the skilled artisan or hereafter discovered. In one exemplary method, BET surface area is measured with a Gemini III 2375 Surface Area Analyzer, using pure nitrogen as the sorbent gas, from Micromeritics Instrument Corporation (Norcross, Ga., USA).

In some embodiments the clay of the antimicrobial composition has a shape factor of less than about 45, or less than about 30. For example, the shape factor may range from about 2 to about 35, from about 2 to about 20, or from about 5 to about 15. A clay having a relatively high shape factor may be considered to be more plate-shaped than a clay having a low shape factor, which may be considered to be more block-shaped.

“Shape factor” as used herein is a measure of an average value (on a weight average basis) of the ratio of mean particle diameter to particle thickness for a population of particles of varying size and shape, as measured using the electrical conductivity method and apparatus described in GB No. 2,240,398, U.S. Pat. No. 5,128,606, EP No. 0 528 078, U.S. Pat. No. 5,576,617, and EP 631 665, and using the equations derived in these publications. For example, in the measurement method described in EP No. 0 528 078, the electrical conductivity of a fully dispersed aqueous suspension of the particles under test is caused to flow through an elongated tube. Measurements of the electrical conductivity are taken between (a) a pair of electrodes separated from one another along the longitudinal axis of the tube, and (b) a pair of electrodes separated from one another across the transverse width of the tube, and by using the difference between the two conductivity measurements, the shape factor of the particulate material under test is determined. “Mean particle diameter” is defined as the diameter of a circle, which has the same area as the largest face of the particle.

Antimicrobial compositions of the present disclosure may also include at least one transition metal compound-either as a separate component of the composition or as a component included within the clay itself. In some embodiments, the clay comprises a transition metal compound. For example, in some embodiments a crystalline structure of the clay may include a transition metal compound. In such embodiments the clay may include a transition metal compound containing a transition metal selected from Ti, V, Mn, Fe, Co, Ni, Cu, Zn, Zr, Mo, Ru, Rh, Pd, Ag, Pt, Au and mixtures thereof.

The transition metal compound may be a compound added to the clay, or the transition metal compound may be the product of a compound or material added to the clay. For example, in some embodiments the transition metal compound is provided by adding a transition metal or an alloy containing the transition metal to the clay, and the transition metal or alloy is converted into the transition metal compound.

In some embodiments the clay may contain an iron compound selected from iron, an alloy containing iron, ammonium iron(II) sulfate [(NH₄)₂Fe(SO₄)₂], ammonium iron (III) sulfate [NH₄Fe(SO₄)₂], iron(III) carbonate [Fe₂(CO₃)₃], iron(II) sulfate [FeSO₄], iron(III) oxide [Fe₂O₃], iron(II) acetate [Fe(CH₃COO)₂], iron(IIl) acetate [Fe(C₂H₃O₂)₃], iron(II) nitrate [Fe(NO₃)₂], iron(II) phosphate [Fe₃(PO₄)₂], iron(II) nitrite [Fe(NO₂)₂], iron(III) sulfate [Fe₂(SO₄)₃], iron(III) chlorate [Fe(C O₃)₃], iron(III) phosphate [FePO₄], iron(III) chloride [FeCl₃], iron(II) sulfate [FeSO₄], iron(III) sulfite [Fe₂(SO₃)₃], iron(II) carbonate [FeCO₃], iron(II) sulfite [FeSO₃], iron(II) chloride [FeCl₂], iron(III) nitrite [Fe(NO₂)₃], iron(II) oxide [FeO], iron(III) nitrate [Fe(NO₃)₃], iron(III) permanganate [Fe(MnO₄)₃], iron(III) sulfide [Fe₂S₃], iron(II) iodate [Fe(O₃)₂], iron(II) perchlorate [Fe(ClO₄)₂], hydrates thereof, and mixtures thereof, just to name a few.

In some embodiments the clay may contain a copper compound selected from copper, an alloy containing copper, copper(II) sulfate [CuSO₄], copper(II) iodide [CuI₂], copper(I) carbonate [Cu₂CO₃], copper(II) phosphate [Cu₃(PO₄)₂], copper(II) nitrate [Cu(NO₃)₂], copper(II) sulfate [CuSO₄], copper(I) sulfate [Cu₂SO₄], copper(II) sulfite [CuSO₃], copper(II) nitrite [Cu(NO₂)₂], copper(II) Iodate [Cu(IO₃)₂], copper (I) oxide [Cu₂O], copper (II) oxide [CuO], copper(I) sulfite [Cu₂SO₃], copper(II) nitrate [Cu(NO₃)₂], copper(II) chlorate [Cu(ClO₃)₂], copper(II) chloride [CuCl₂], copper(II) Hydrogen carbonate [Cu(HCO₃)₂], copper(I) nitrate [CuNO₃], copper(I) phosphate [Cu₃PO₄], copper(II) chlorite [Cu(ClO₂)₂], copper(II) nitrate [Cu(NO₃)₂], copper(I) chlorate [CuClO₃], copper(I) nitrite [CuNO₂], copper(II) phosphite [Cu₃(PO₃)₂], copper(II) hydrogen sulfate [Cu(HSO₄)₂], copper(II) permanganate [Cu(MnO₄)₂], hydrates thereof, and mixtures thereof, just to name a few.

In still other embodiments the antimicrobial composition may include a clay containing an iron compound and/or a copper compound—wherein a crystalline structure of the clay may include the iron compound and/or the copper compound.

Embodiments of the present disclosure also include methods involving a step of choosing a clay containing a transition metal compound, and then mixing the chosen clay with an aluminum compound. In some embodiments the step of choosing a clay containing a transition metal compound is performed such that the chosen clay possesses certain characteristics such as, for example: (i) a certain proportion of the transition metal atom or metal ion in the clay (e.g., 3-4% by mass of the transition metal atom in the clay); (ii) a certain pH of the clay (e.g., pH of the clay being less than or equal to about 5); and/or (iii) a certain oxidation-reduction potential (ORP) of the clay (e.g., ORP of the clay ranging from about 300 mV to about 800 mV). Some embodiments include methods involving choosing a clay containing a transition metal selected from Ti, V, Mn, Fe, Co, Ni, Cu, Zn, Zr, Mo, Ru, Rh, Pd, Ag, Pt, Au or mixtures thereof.

In some embodiments the antimicrobial composition may include the clay, the aluminum compound, and a transition metal compound as an additional component. In still other embodiments the antimicrobial composition may contain at least two transition metal compounds and/or at least two aluminum compounds. Thus, antimicrobial compositions of the present disclosure include compositions contain a binary mixture (clay and aluminum compound), a ternary mixture (clay, aluminum compound and transition metal compound), and more complex mixtures containing a plurality of at least one of the clay, the aluminum compound and the transition metal compound—as well as other components and additives.

For example, antimicrobial compositions of the present disclosure may include (as a separate component from the clay and the aluminum compound) a transition metal compound containing a transition metal selected from Ti, V, Mn, Fe, Co, Ni, Cu, Zn, Zr, Mo, Ru, Rh, Pd, Ag, Pt, Au and mixtures thereof, just to name a few.

In some embodiments the antimicrobial composition may also contain (as a separate component from the clay and the aluminum compound) an iron compound selected from the group consisting of iron, an alloy containing iron, ammonium iron(II) sulfate [(NH₄)₂Fe(SO₄)₂], ammonium iron (II) sulfate [NH₄Fe(SO₄)₂], iron(III) carbonate [Fe₂(CO₃)₃], iron(II) sulfate [FeSO₄], iron(III) oxide [Fe₂O₃], iron(II) acetate [Fe(CH₃COO)₂], iron(III) acetate [Fe(C₂H₃O₂)₃], iron(II) nitrate [Fe(NO₃)₂], iron(II) phosphate [Fe₃(PO₄)₂], iron(II) nitrite [Fe(NO₂)₂], iron(III) sulfate [Fe₂(SO₄)₃], iron(II) chlorate [Fe(CO₃)₃], iron(III) phosphate [FePO₄], iron(III) chloride [FeCl₃], iron(II) sulfate [FeSO₄], iron(III) sulfite [Fe₂(SO₃)₃], iron(II) carbonate [FeCO₃], iron(II) sulfite [FeSO₃], iron(II) chloride [FeCl₂], iron(III) nitrite [Fe(NO₂)₃], iron(II) oxide [FeO], iron(II) nitrate [Fe(NO₃)₃], iron(III) permanganate [Fe(MnO₄)₃], iron(III) sulfide [Fe₂S₃], iron(II) iodate [Fe(IO₃)₂], iron(II) perchlorate [Fe(ClO₄)₂], hydrates thereof, and mixtures thereof, just to name a few.

In other embodiments the antimicrobial composition may also contain (as a separate component from the clay and the aluminum compound) a copper compound selected from copper, an alloy containing copper, copper(II) sulfate [CuSO₄], copper(II) iodide [CuI₂], copper(I) carbonate [Cu₂CO₃], copper(II) phosphate [Cu₃(PO₄)₂], copper(II) nitrate [Cu(NO₃)₂], copper(II) sulfate [CuSO₄], copper(I) sulfate [Cu₂SO₄], copper(II) sulfite [CuSO₃], copper(II) nitrite [Cu(NO₂)₂], copper(II) lodate [Cu(IO₃)₂], copper (II) oxide [Cu₂O], copper (II) oxide [CuO], copper(I) sulfite [Cu₂SO₃], copper(II) nitrate [Cu(NO₃)₂], copper(II) chlorate [Cu(Cu(ClO₃)₂], copper(II) chloride [CuCl₂], copper(II) Hydrogen carbonate [Cu(HCO₃)₂], copper(I) nitrate [CuNO₃], copper(I) phosphate [Cu₃PO₄], copper(II) chlorite [Cu(CO₂)₂], copper(II) nitrate [Cu(NO₃)₂], copper(I) chlorate [CuClO₃], copper(I) nitrite [CuNO₂], copper(II) phosphite [Cu₃(PO₃)₂], copper(II) hydrogen sulfate [Cu(HSO₄)₂], copper(II) permanganate [Cu(MnO₄)₂], hydrates thereof, and mixtures thereof, just to name a few.

In some embodiments the antimicrobial composition may include (as separate components from the clay and the aluminum compound) an iron compound and a copper compound.

For example, in some embodiments the antimicrobial composition includes a transition metal compound as a separation component, such that the composition is a ternary mixture in which: the clay is a kaolinic clay; the transition metal compound comprises at least one selected from iron (II) sulfate, an iron (III) sulfate, a copper (I) sulfate, and a copper (II) sulfate; and the aluminum compound comprises an aluminum sulfate, a potassium aluminum sulfate, or a mixture thereof.

In some embodiments the proportion of the aluminum compound, the transition metal compound, or both, may be selected to attain a high level of antimicrobial activity. For example, in some embodiments a proportion of the aluminum compound and/or the transition metal compound (if separately present) may range from about 0.10 wt. % to about 5.0 wt. %, relative to a total weight of the clay. In other embodiments the proportion of the aluminum compound and/or the transition metal compound may range from about 0.25 wt. % to about 4.5 wt. %, or from about 0.3 wt. % to about 2.5 wt. %, or from about 0.3 wt. % to about 2 wt. %, or from about 0.5 wt. % to about 1.8 wt. %, relative to a total weight of the clay. In still other embodiments the proportion of the aluminum compound and/or the transition metal compound may fall well outside of these ranges. For example, in some embodiments the proportion of the aluminum compound and/or the transition metal compound may range from about 1 wt. % to about 500 wt. %, relative to a total weight of the clay.

In some embodiments the antimicrobial composition includes both of an aluminum compound and a transitional metal compound as separate components, such that a proportion of the transition metal compound ranges from 0.10 wt % to 5.0 wt %, and a proportion of the aluminum compound ranges from 0.10 wt % to 5.0 wt %, relative to the total weight of the clay. In still other embodiments a gram weight ratio of the transition metal compound to the aluminum compound in the antimicrobial composition ranges from 0.01:99.99 to 99.99:0.01.

Depending upon the form of the antimicrobial composition, in some embodiments the composition may include other components such as a liquid or other additive. For example, in some embodiments the antimicrobial composition may further contain a pharmaceutically acceptable liquid. In still other embodiments the antimicrobial composition may further contain an aqueous liquid or a solvent.

The term “aqueous liquid” as used herein describes a liquid containing water and at least one solvent. The term “solvent” as used herein means an organic solvent. For example, in some embodiments the antimicrobial composition may contain at least one solvent selected from water, an ether-containing solvent, an alcohol-containing solvent, an amine-containing solvent, an acid-containing solvent, an ester-containing solvent, a ketone-containing solvent, an aromatic hydrocarbon-containing solvent, an aliphatic hydrocarbon-containing solvent, a polar protic solvent, a polar aprotic solvent, and mixtures thereof, just to name a few. Solvents of the antimicrobial composition may also be compounds of mixed character, such as aliphatic-aromatic compounds, alcohol-ester compounds, alcohol-ether compounds, to name a few. Solvents of the antimicrobial composition may also be halogenated compounds such as halogenated aromatic compounds and halogenated aliphatic compounds.

In some embodiments the antimicrobial composition may include at least one solvent selected from acetone, acetonitrile, anisole, benzene, benzonitrile, benzyl alcohol, 1,3-butanediol, 2-butanone, tert-butanol, 1-butanol, 2-butanol, 2-(2-butoxyethoxy)ethyl acetate, 2-butoxyethyl acetate, butyl acetate, tert-butyl aceto acetate, tert-butyl methyl ether, carbon disulfide, carbon tetrachloride, chlorobenzene, 1-chlorobutane, chloroform, cyclohexane, cyclopentane, cyclopentyl methyl ether, decane, dibutyl ether, 1,2-dichlorobenzene, 1,2-dichloroethane, dichloromethane, diethyl ether, diethylene glycol butyl ether, diethylene glycol diethyl ether, diethylene glycol dimethyl ether, diethylene glycol monoethyl ether, diethylene glycol monoethyl ether acetate, diisopropyl ether, N,N-diisopropylethylamine, 1,2-dimethoxyethane, dimethyl carbonate, dimethyl sulfoxide, N,N-dimethylacetamide, 1,4-dioxane, 1,3-dioxolane, dodecane, ethanol, 2-ethoxyethanol, ethyl 3-ethoxyproprionate, ethyl acetate, ethylbenzene, ethylene carbonate, ethylene glycol, ethylene glycol butyl ether, ethylene glycol diethyl ether, 2-ethylhexyl acetate, formamide, glycerol, heptane, 2-heptanone, hexadecane, hexane, hexanol, isopentyl acetate, isopropyl acetate, isopropyl alcohol, methanol, 2-methoxyethanol, 2-methoxyethyl acetate, 1-methoxy-2-propanol, methyl acetate, methyl formate, 2-methylbutane, isoamyl alcohol, methylcyclohexane, 5-methyl-2-hexanone, 4-methyl-2-pentanone, isobutyl alcohol, 1-methyl-2-pyrrolidinone, 2-methyltetrahydrofuran, nitrobenzene, nitromethane, nonane, octane, 1-octanol, pentane, 1-pentanol, 2-pentanone, 3-pentanone, petroleum ether, piperidine, 1-propanol, 2-propanol, 2-propoxyethanol, propyl acetate, propylene carbonate, pyridine, 1,1,2,2-tetrachloroethane, tetrachloroethylene, tetrahydrofuran, toluene, 1,2,4-trichlorobenzene, 2,2,4-trimethylpentane, water, m-xylene, o-xylene, p-xylene, and mixtures thereof, just to name a few.

In some embodiments in which the antimicrobial composition includes an aqueous liquid, the antimicrobial composition may exist as a paste. In still other embodiments the antimicrobial composition may exists as an aqueous or non-aqueous dispersion, or as an aqueous or non-aqueous solution.

In some embodiments the antimicrobial composition may further include at least one additive selected from a transition metal compound, a reducing agent, an antioxidant, and oxygen scavenger, a filler, a dispersant, an organic polymer, a pigment, a therapeutic agent and an antiseptic, just to name a few. For example, in some embodiments the antimicrobial composition may be in a form wherein it is in contact with a polymer material.

In some embodiments of the present disclosure the antimicrobial composition is adapted to function as an antibacterial composition. For example, in some embodiments the antimicrobial composition is adapted to function as an antibacterial composition effective in treating a bacterial skin infection caused by one or more bacteria selected from the group consisting of E. coli, ESBL E. coli, M. marinum, Mycobacterium ulcerans, MRSA, M. smegmatis, Pseudomonas aeruginosa, Salmonella typhimurium, Staphylococcus epidermidis, S. aureus, and Streptococcus sp.

### Methods for Producing Antimicrobial Compositions

Embodiments of the present disclosure also include methods for producing antimicrobial compositions. For example, in some embodiments the antimicrobial composition is prepared by combining the clay, the aluminum compound, and the optional transition metal compound.

In some embodiments the method of producing the antimicrobial composition also includes a step of reducing a particle size of the clay to form a fine clay having a particle size distribution such that greater than about 20% by weight and less than about 60% by weight of particles of the fine clay have a particle size of less than 0.25 microns as measured by Sedigraph. Such a step of reducing the particle size may occur prior to combining the fine clay with the aluminum compound and the optional transition metal compound, or may occur to a solid obtained after combining the clay, the aluminum compound and the optional transition metal compound.

For example, in some embodiments the method of producing the antimicrobial composition includes the steps of reducing the particle size of the clay, and reducing the particle size of the aluminum compound, the transition metal compound, or both, prior to combining the clay with the aluminum compound, the transition metal compound, or both.

In some embodiments the method of producing the antimicrobial composition involves combining the clay, the aluminum compound, and the optional transition metal compound, to obtain a dispersion comprising the clay, the aluminum compound and the optional transition metal compound. For example, the method may include the steps of: forming a dispersion comprising the clay, the aluminum compound, and the optional transition metal compound; and adjusting the pH of the dispersion, adjusting the oxidation-reduction potential (ORP) of the dispersion, or adjusting the pH and the oxidation-reduction potential (ORP) of the dispersion, to obtain the antimicrobial composition.

In still other embodiments the method may involve combining the clay, the aluminum compound and the optional transition metal compound, in the presence of a pharmaceutically acceptable liquid, to obtain the antimicrobial composition. For example, the may include the steps of: forming a dispersion comprising the clay, the aluminum compound, and optionally a transition metal compound; and processing the dispersion into a paste. In still other embodiments the antimicrobial composition may be formed by blending the clay, the aluminum compound and the optional transition metal compound into a polymer to obtain a polymer-based microbial composition. For instance, the clay, the aluminum compound and the optional transitional metal compound may be blended to form a mixture which is then applied to the surface of a polymer film to obtain a polymer-based microbial composition.

### Methods of Using Antimicrobial Composition

Embodiments of the present disclosure also include methods of reducing bacterial viability using the antimicrobial compositions described herein. For example, the present disclosure includes a method for reducing bacterial viability of one or more bacteria, which involves applying a bactericidal effective amount of the antimicrobial composition to the one or more bacteria.

In some embodiments the method for reducing the bacterial viability involves applying a bactericidal effective amount of an antimicrobial composition in which: the clay is a kaolinic clay; the aluminum compound comprises an aluminum sulfate, a potassium aluminum sulfate, or a mixture thereof; and the antimicrobial composition optionally comprises at least one transition metal compound selected from the group consisting of an iron (II) sulfate, an iron (III) sulfate, a copper (I) sulfate, and a copper (II) sulfate.

Embodiments of the present disclosure also include methods for treating or preventing bacterial infections in or on a subject. Treatable bacterial infections may be caused by one or more bacteria selected from E. coli, ESBL E. coli, M. marinum, Mycobacterium ulcerans, MRSA, M. smegmatis, Pseudomonas aeruginosa, Salmonella typhimurium, Staphylococcus epidermidis, S. aureus, and Streptococcus sp, just to name a few. In some embodiments the method involves administering a bactericidal effective amount of the antimicrobial composition to a subject in need of treatment at a site of the bacterial infection.

In some embodiments the method for treating or preventing bacterial infections involves administering a bactericidal effective amount of the antimicrobial composition in which: the clay is a kaolinic clay; the aluminum compound comprises an aluminum sulfate, a potassium aluminum sulfate, or a mixture thereof; and the antimicrobial composition optionally comprises at least one transition metal compound selected from the group consisting of an iron (II) sulfate, an iron (III) sulfate, a copper (I) sulfate, and a copper (II) sulfate.

In some embodiments the antimicrobial activity of the antimicrobial composition may be enhanced by the presence of both the aluminum compound and a transitional metal compound. For example, in some embodiments the antibacterial activity of an antibacterial composition containing the clay, the aluminum compound and a transitional metal compound is surprisingly greater than an expected antibacterial activity-based upon the activity of one composition containing the clay and the aluminum compound alone, and another composition containing the clay and the transition metal compound alone.

Antimicrobial compositions of the present disclosure may be used to treat bacterial skin infections and diseases, including infections and diseases caused by antibiotic resistant bacteria. For example, compositions of the present disclosure may be used to treat infections and diseases caused by methicillin resistant Staph. Aureus (MRSA) as well as gram positive and acid fast bacteria.

Without being bound to any particular theory, it is possible that the surprisingly greater antibacterial activity of antibacterial clays containing both the aluminum compound and the transition metal compound may be due to an ability of aluminum ions (Al³⁺) to alter the functioning of the FhuA and TonB cell membrane proteins of E. coli-thereby enhancing the influx of transition metal ions (e.g., Fe²⁺/Fe³⁺) into E. coli cells leading to oxidative stress.

A possible mechanism explaining the enhanced antibacterial activity of antimicrobial compositions of the present disclosure involves the presence of transition metal compounds, such as soluble iron or copper compounds, which allows transition metal ions (e.g., Fe²⁺/Fe³⁺) to bind to siderophores such as a hydroxamate siderophore. The resulting transition metal-bound-sideophores may then interact with surface proteins on the cell wall and appropriate active transport systems on the cell wall membrane of E. coli cells-leading to a conformation shift in the beta-barrel proteins allowing, for example, iron-bound-sideophores to be transported through the cell wall and ultimately into the intercellular matrix. Fe³⁺ actively transported into the intercellular matrix is then converted into Fe²⁺ via electron cleaving. The uptake of Fe³⁺ into the cell is regulated by the ferric uptake regulatory (Fur) protein, a repressor. In excess, Fe²⁺ also binds to the transcriptional activator FNR, the inducible control for anaerobic respiration. Thus, mechanisms for cleaving Fe³⁺ into the usable Fe²⁺ are disabled, despite the presence of such ions, and anaerobic respiration factors are transcribed despite the presence of oxygen.

In this possible mechanism the presence of aluminum compounds, such as soluble Al³⁺ compounds, further enhances the influx of Fe³⁺ into E. coli cells by binding to hydroxamate siderophores. The resulting aluminum-bound-sideophore may then interact with Fe³⁺ specific cell wall proteins triggering a partial conformational change in the beta barrel structure. However, the Al³⁺ siderophore is unable to pass through the cell wall without the active transport facilitated by the energy from TonB. This creates a gap in the cell wall proteins large enough to allow an unregulated influx of unbound Fe³⁺ ions. Such unregulated influx of Fe³⁺ ions, in conjunction with the repressed Fur regulated genes and the induced fnr gene (both resulting from the excess of Fe²⁺ passively entering the intercellular matrix from the environment) triggers oxidative stress, and transcription of anaerobic transcription factors under aerobic conditions.

Applications of the antimicrobial compositions of the present disclosure include their use in the areas of pharmaceuticals, control of the MRSA epidemic, wound care, sterility wraps, cosmetics and hygiene products such as makeup, acne treatments and deodorants, athletic gear products such as odor control clothing, agricultural products, antibacterial coatings, and methods of treating water, just to name a few.

### Exemplary Embodiments

Embodiment [1] of the present disclosure relates to an antimicrobial composition, comprising: a clay; and an aluminum compound, wherein: a pH of the antimicrobial composition is less than or equal to 5; and an oxidation-reduction potential (ORP) of the antimicrobial composition ranges from about 300 mV to about 800 mV.

Embodiment [2] of the present disclosure relates to the antimicrobial composition of Embodiment [1], wherein at least one of the following conditions is satisfied: the clay comprises a transition metal compound; the composition further comprises a transition metal compound.

Embodiment [3] of the present disclosure relates to the antimicrobial composition of Embodiments [1]-[2], wherein at least one of the following conditions is satisfied: the clay comprises a transition metal compound containing a transition metal selected from the group consisting of Ti, V, Mn, Fe, Co, Ni, Cu, Zn, Zr, Mo, Ru, Rh, Pd, Ag, Pt, Au and mixtures thereof; the composition further comprises a transition metal compound containing a transition metal selected from the group consisting of Ti, V, Mn, Fe, Co, Ni, Cu, Zn, Zr, Mo, Ru, Rh, Pd, Ag, Pt, Au and mixtures thereof.

Embodiment [4] of the present disclosure relates to the antimicrobial composition of Embodiments [1]-[3], wherein the clay is selected from the group consisting of a bentonite clay, a chlorite clay, an illite clay, a kaolinic clay, a montmorillonite clay, a rectorite clay, a smectite clay, and mixtures thereof.

Embodiment [5] of the present disclosure relates to the antimicrobial composition of Embodiments [1]-[4], wherein the clay is a clay having a particle size distribution such that greater than about 20% by weight and less than about 60% by weight of particles of the clay have a particle size of less than 0.25 microns as measured by Sedigraph.

Embodiment [6] of the present disclosure relates to the antimicrobial composition of Embodiments [1]-[5], wherein a d₅₀ of the aluminum compound ranges from 1 nm to 1000 nm.

Embodiment [7] of the present disclosure relates to the antimicrobial composition of Embodiments [1]-[6], wherein the aluminum compound is selected from the group consisting of aluminum, an alloy containing aluminum, aluminum acetate [Al(C₂H₃O₂)₃], aluminum sulfate [Al₂(SO₄)₃], potassium aluminum sulfate [KAl(SO₄)₂], aluminum carbonate [Al₂(CO₃)₃], aluminum sulfite [Al₂(SO₃)₃], aluminum oxide [Al₂O₃], aluminum chlorate [Al(ClO₃)₃], aluminum sulfide [Al₂S₃], aluminum nitrate [Al(NO₃)₃], aluminum permanganate [Al(MnO₄)₃], aluminum hydrogen carbonate [Al(HCO₃)₃], aluminum phosphate [AlPO₄], aluminum oxalate [Al₂(C₂O₄)₃], aluminum hydrogen phosphate [Al₂(HPO₄)₃], aluminum thiosulfate [Al₂(S₂O₃)₃], aluminum chlorite [Al(ClO₂)₃], aluminum hydrogen sulfate [Al(HSO₄)₃], aluminum dihydrogen phosphate [Al(H₂PO₄)₃], aluminum phosphite [AlPO₃], aluminum potassium sulfate [KAl(SO₄)₂], hydrates thereof, and mixtures thereof.

Embodiment [8] of the present disclosure relates to the antimicrobial composition of Embodiments [1]-[7], wherein a crystalline structure of the clay includes a transition metal compound.

Embodiment [9] of the present disclosure relates to the antimicrobial composition of Embodiments [1]-[8], wherein the clay comprises an iron compound, a copper compound, or a mixture thereof.

Embodiment [10] of the present disclosure relates to the antimicrobial composition of Embodiments [1]-[9], wherein the clay comprises an iron compound selected from the group consisting of iron, an alloy containing iron, ammonium iron(II) sulfate [(NH₄)₂Fe(SO₄)₂], ammonium iron (III) sulfate [NH₄Fe(SO₄)₂], iron(III) carbonate [Fe₂(CO₃)₃], iron(II) sulfate [FeSO₄], iron(III) oxide [Fe₂O₃], iron(II) acetate [Fe(CH₃COO)₂], iron(III) acetate [Fe(C₂H₃O₂)₃], iron(II) nitrate [Fe(NO₃)₂], iron(II) phosphate [Fe₃(PO₄)₂], iron(II) nitrite [Fe(NO₂)₂], iron(III) sulfate [Fe₂(SO₄)₃], iron(III) chlorate [Fe(ClO₃)₃], iron(III) phosphate [FePO₄], iron(III) chloride [FeCl₃], iron(II) sulfate [FeSO₄], iron(III) sulfite [Fe₂(SO₃)₃], iron(II) carbonate [FeCO₃], iron(II) sulfite [FeSO₃], iron(II) chloride [FeCl₂], iron(III) nitrite [Fe(NO₂)₃], iron(II) oxide [FeO], iron(III) nitrate [Fe(NO₃)₃], iron(III) permanganate [Fe(MnO₄)₃], iron(III) sulfide [Fe₂S₃], iron(II) iodate [Fe(IO₃)₂], iron(II) perchlorate [Fe(ClO₄)₂], hydrates thereof, and mixtures thereof.

Embodiment [11] of the present disclosure relates to the antimicrobial composition of Embodiments [1]-[10], wherein the clay comprises a copper compound selected from the group consisting of copper, an alloy containing copper, copper(II) sulfate [CuSO₄], copper(II) iodide [CuI₂], copper(I) carbonate [Cu₂CO₃], copper(II) phosphate [Cu₃(PO₄)₂], copper(II) nitrate [Cu(NO₃)₂], copper(II) sulfate [CuSO₄], copper(I) sulfate [Cu₂SO₄], copper(II) sulfite [CuSO₃], copper(II) nitrite [Cu(NO₂)₂], copper(II) lodate [Cu(IO₃)₂], copper (I) oxide [Cu₂O], copper (II) oxide [CuO], copper(I) sulfite [Cu₂SO₃], copper(II) nitrate [Cu(NO₃)₂], copper(II) chlorate [Cu(ClO₃)₂], copper(II) chloride [CuCl₂], copper(II) Hydrogen carbonate [Cu(HCO₃)₂], copper(I) nitrate [CuNO₃], copper(I) phosphate [Cu₃PO₄], copper(II) chlorite [Cu(ClO₂)₂], copper(II) nitrate [Cu(NO₃)₂], copper(I) chlorate [CuClO₃], copper(I) nitrite [CuNO₂], copper(II) phosphite [Cu₃(PO₃)₂], copper(II) hydrogen sulfate [Cu(HSO₄)₂], copper(II) permanganate [Cu(MnO₄)₂], hydrates thereof, and mixtures thereof.

Embodiment [12] of the present disclosure relates to the antimicrobial composition of Embodiments [1]-[11], further comprising a transition metal compound.

Embodiment [13] of the present disclosure relates to the antimicrobial composition of Embodiments [1]-[12], wherein at least one of the following conditions is satisfied: the antimicrobial composition comprises at least two transition metal compounds; the antimicrobial composition comprises at least two aluminum compounds.

Embodiment [14] of the present disclosure relates to the antimicrobial composition of Embodiments [1]-[13], further comprising a transition metal compound containing a transition metal selected from the group consisting of Ti, V, Mn, Fe, Co, Ni, Cu, Zn, Zr, Mo, Ru, Rh, Pd, Ag, Pt, Au and mixtures thereof.

Embodiment [15] of the present disclosure relates to the antimicrobial composition of Embodiments [1]-[14], further comprising an iron compound selected from the group consisting of iron, an alloy containing iron, ammonium iron(II) sulfate [(NH₄)₂Fe(SO₄)₂], ammonium iron (III) sulfate [NH₄Fe(SO₄)₂], iron(III) carbonate [Fe₂(CO₃)₃], iron(II) sulfate [FeSO₄], iron(II) oxide [Fe₂O₃], iron(II) acetate [Fe(CH₃COO)₂], iron(III) acetate [Fe(C₂H₃O₂)₃], iron(II) nitrate [Fe(NO₃)₂], iron(II) phosphate [Fe₃(PO₄)₂], iron(II) nitrite [Fe(NO₂)₂], iron(III) sulfate [Fe₂(SO₄)₃], iron(III) chlorate [Fe(ClO₃)₃], iron(III) phosphate [FePO₄], iron(III) chloride [FeCl₃], iron(II) sulfate [FeSO₄], iron(III) sulfite [Fe₂(SO₃)₃], iron(II) carbonate [FeCO₃], iron(II) sulfite [FeSO₃], iron(II) chloride [FeCl₂], iron(III) nitrite [Fe(NO₂)₃], iron(II) oxide [FeO], iron(III) nitrate [Fe(NO₃)₃], iron(III) permanganate [Fe(MnO₄)₃], iron(III) sulfide [Fe₂S₃], iron(II) iodate [Fe(IO₃)₂], iron(II) perchlorate [Fe(ClO₄)₂], hydrates thereof, and mixtures thereof.

Embodiment [16] of the present disclosure relates to the antimicrobial composition of Embodiments [1]-[15], further comprising a copper compound selected from the group consisting of copper, an alloy containing copper, copper(II) sulfate [CuSO₄], copper(II) iodide [CuI₂], copper(I) carbonate [Cu₂CO₃], copper(II) phosphate [Cu₃(PO₄)₂], copper(II) nitrate [Cu(NO₃)₂], copper(II) sulfate [CuSO₄], copper(I) sulfate [Cu₂SO₄], copper(II) sulfite [CuSO₃], copper(II) nitrite [Cu(NO₂)₂], copper(II) lodate [Cu(IO₃)₂], copper (I) oxide [Cu₂O], copper (II) oxide [CuO], copper(I) sulfite [Cu₂SO₃], copper(II) nitrate [Cu(NO₃)₂], copper(II) chlorate [Cu(ClO₃)₂], copper(II) chloride [CuCl₂], copper(II) Hydrogen carbonate [Cu(HCO₃)₂], copper(I) nitrate [CuNO₃], copper(I) phosphate [Cu₃PO₄], copper(II) chlorite [Cu(ClO₂)₂], copper(II) nitrate [Cu(NO₃)₂], copper(I) chlorate [CuClO₃], copper(I) nitrite [CuNO₂], copper(II) phosphite [Cu₃(PO₃)₂], copper(II) hydrogen sulfate [Cu(HSO₄)₂], copper(II) permanganate [Cu(MnO₄)₂], hydrates thereof, and mixtures thereof.

Embodiment [17] of the present disclosure relates to the antimicrobial composition of Embodiments [1]-[16], further comprising a transition metal compound, wherein the aluminum compound is selected from the group consisting of aluminum, an alloy containing aluminum, aluminum acetate [Al(C₂H₃O₂)₃], aluminum sulfate [Al₂(SO₄)₃], potassium aluminum sulfate [KAl(SO₄)₂], aluminum carbonate [Al₂(CO₃)₃], aluminum sulfite [Al₂(SO₃)₃], aluminum oxide [Al₂O₃], aluminum chlorate [Al(ClO₃)₃], aluminum sulfide [Al₂S₃], aluminum nitrate [Al(NO₃)₃], aluminum permanganate [Al(MnO₄)₃], aluminum hydrogen carbonate [Al(HCO₃)₃], aluminum phosphate [AlPO₄], aluminum oxalate [Al₂(C₂O₄)₃], aluminum hydrogen phosphate [Al₂(HPO₄)₃], aluminum thiosulfate [Al₂(S₂O₃)₃], aluminum chlorite [Al(ClO₂)₃], aluminum hydrogen sulfate [Al(HSO₄)₃], aluminum dihydrogen phosphate [Al(H₂PO₄)₃], aluminum phosphite [AlPO₃], aluminum potassium sulfate [KAl(SO₄)₂], hydrates thereof, and mixtures thereof.

Embodiment [18] of the present disclosure relates to the antimicrobial composition of Embodiments [1]-[17], further comprising an iron compound, a copper compound, or a mixture thereof.

Embodiment [19] of the present disclosure relates to the antimicrobial composition of Embodiments [1]-[18], further comprising a transition metal compound, wherein: the clay is a kaolinic clay; the transition metal compound comprises at least one selected from the group consisting of an iron (II) sulfate, an iron (III) sulfate, a copper (I) sulfate, and a copper (II) sulfate; and the aluminum compound comprises an aluminum sulfate, a potassium aluminum sulfate, or a mixture thereof.

Embodiment [20] of the present disclosure relates to the antimicrobial composition of Embodiments [1]-[19], wherein the pH of the antimicrobial composition ranges from 2 to 5.

Embodiment [21] of the present disclosure relates to the antimicrobial composition of Embodiments [1]-[20], wherein a proportion of the aluminum compound ranges from 0.10 wt % to 5.0 wt %, relative to a total weight of the clay.

Embodiment [22] of the present disclosure relates to the antimicrobial composition of Embodiments [1]-[21], further comprising a transition metal compound, wherein: a proportion of the transition metal compound ranges from 0.10 wt % to 5.0 wt %, relative to a total weight of the clay; and a proportion of the aluminum compound ranges from 0.10 wt % to 5.0 wt %, relative to the total weight of the clay.

Embodiment [23] of the present disclosure relates to the antimicrobial composition of Embodiments [1]-[22], further comprising a transition metal compound, wherein a gram weight ratio of the transition metal compound to the aluminum compound ranges from 0.01:99.99 to 99.99:0.01.

Embodiment [24] of the present disclosure relates to the antimicrobial composition of Embodiments [1]-[23], further comprising a pharmaceutically acceptable liquid.

Embodiment [25] of the present disclosure relates to the antimicrobial composition of Embodiments [1]-[24], further comprising an aqueous liquid.

Embodiment [26] of the present disclosure relates to the antimicrobial composition of Embodiments [1]-[25], further comprising an aqueous liquid, wherein the antimicrobial composition exists as a paste.

Embodiment [27] of the present disclosure relates to the antimicrobial composition of Embodiments [1]-[26], further comprising at least one selected from the group consisting of a transition metal compound, a reducing agent, an antioxidant, an oxygen scavenger, a filler, a dispersant, an organic polymer, a pigment, a therapeutic agent and an antiseptic.

Embodiment [28] of the present disclosure relates to the antimicrobial composition of Embodiments [1]-[27], wherein the clay comprises: 0.5-5.0 wt % of Fe₂O₃; 0.0-1.0 wt % of MgO; 10.0-50.0 wt % of Al₂O₃; 10.0-50.0 wt % of SiO₂; 1.0-5.0 wt % of TiO₂; 0.1-1.0 wt % of CaO; 0.1-2.0 wt % of Na₂O; 0.1-1.0 wt % of K₂O; 0.05-1.0 wt % of P₂O₅; 0.0-5.0 wt % of Horiba S; and 0.01-7.0 wt % of FeS₂, relative to a total weight of the clay.

Embodiment [29] of the present disclosure relates to the antimicrobial composition of Embodiments [1]-[28], which is adapted to function as an antibacterial composition.

Embodiment [30] of the present disclosure relates to the antimicrobial composition of Embodiments [1]-[29], which is adapted to function as an antibacterial composition effective in treating a bacterial skin infection caused by one or more bacteria selected from the group consisting of E. coli, ESBL E. coli, M. marinum, Mycobacterium ulcerans, MRSA, M. smegmatis, Pseudomonas aeruginosa, Salmonella typhimurium, Staphylococcus epidermidis, S. aureus, and Streptococcus sp.

Embodiment [31] of the present disclosure relates to the antimicrobial composition of Embodiments [1]-[30], wherein the antimicrobial composition is in contact with a polymer material.

Embodiment [32] of the present disclosure relates to a method for producing the antimicrobial composition of claim 1, the method comprising combining the clay and the aluminum compound to obtain the antimicrobial composition.

Embodiment [33] of the present disclosure relates to the method of Embodiment [32], comprising combining the clay, the aluminum compound and a transitional metal compound to obtain the antimicrobial composition.

Embodiment [34] of the present disclosure relates to the method of Embodiments [32]-[33], further comprising reducing a particle size of the clay, to form a fine clay having a particle size distribution such that greater than about 20% by weight and less than about 60% by weight of particles of the fine clay have a particle size of less than 0.25 microns as measured by Sedigraph, prior to combining the fine clay with the aluminum compound.

Embodiment [35] of the present disclosure relates to the method of Embodiments [32]-[34], further comprising: reducing a particle size of the clay; and reducing a particle size of the aluminum compound, a transition metal compound, or both, prior to combining the clay with the aluminum compound, the transition metal compound, or both.

Embodiment [36] of the present disclosure relates to the method of Embodiments [32]-[35], comprising combining the clay, the aluminum compound, and a transition metal compound, to obtain a dispersion comprising the clay, the aluminum compound and the transition metal compound.

Embodiment [37] of the present disclosure relates to the method of Embodiments [32]-[36], comprising: forming a dispersion comprising the clay, the aluminum compound, and a transition metal compound; and adjusting the pH of the dispersion, adjusting the oxidation-reduction potential (ORP) of the dispersion, or adjusting the pH and the oxidation-reduction potential (ORP) of the dispersion.

Embodiment [38] of the present disclosure relates to the method of Embodiments [32]-[37], comprising combining the clay, the aluminum compound and optionally a transition metal compound, in the presence of a pharmaceutically acceptable liquid, to obtain the antimicrobial composition.

Embodiment [39] of the present disclosure relates to the method of Embodiments [32]-[38], comprising: forming a dispersion comprising the clay, the aluminum compound, and optionally a transition metal compound; and processing the dispersion into a paste.

Embodiment [40] of the present disclosure relates to the method of Embodiments [32]-[39], comprising blending the clay, the aluminum compound and optionally a transition metal compound in to a polymer to obtain the microbial composition.

Embodiment [41] of the present disclosure relates to the method of Embodiments [32]-[40], comprising: combining the clay, the aluminum compound and optionally a transition metal compound to obtain a mixture; and applying the mixture to the surface of a polymer film.

Embodiment [42] of the present disclosure relates to a method for reducing bacterial viability of one or more bacteria, the method comprising applying a bactericidal effective amount of the antimicrobial composition of claim 1 to the one or more bacteria.

Embodiment [43] of the present disclosure relates to the method of Embodiment [42], wherein: the clay is a kaolinic clay; the aluminum compound comprises an aluminum sulfate, a potassium aluminum sulfate, or a mixture thereof; and the antimicrobial composition optionally comprises at least one transition metal compound selected from the group consisting of an iron (II) sulfate, an iron (III) sulfate, a copper (I) sulfate, and a copper (II) sulfate.

Embodiment [44] of the present disclosure relates to a method for treating or preventing a bacterial infection in or on a subject, in which the bacterial infection is caused by one or more bacteria selected from the group consisting of E. coli, ESBL E. coli, M. marinum, Mycobacterium ulcerans, MRSA, M. smegmatis, Pseudomonas aeruginosa, Salmonella typhimurium, Staphylococcus epidermidis, S. aureus, and Streptococcus sp, the method comprising administering a bactericidal effective amount of the antimicrobial composition of claim 1 to a subject in need thereof at a site of the bacterial infection.

Embodiment [45] of the present disclosure relates to the method of Embodiment [44], wherein: the clay is a kaolinic clay; the aluminum compound comprises an aluminum sulfate, a potassium aluminum sulfate, or a mixture thereof; and the antimicrobial composition optionally comprising at least one transition metal compound selected from the group consisting of an iron (II) sulfate, an iron (III) sulfate, a copper (I) sulfate, and a copper (II) sulfate.

### EXAMPLES

The following examples are provided for illustration purposes only and in no way limit the scope of the present disclosure. Embodiments of the present disclosure may employ the use of different or additional components compared to the materials illustrated below, such as other antimicrobial compositions containing different aluminum compounds and different transition metal compounds, as well as additional components and additives. Embodiments of the present disclosure may also employ the use of different process conditions than the conditions illustrated below for the preparation of antimicrobial compositions. Embodiments of the present disclosure may also employ different methods for reducing microbial or bacterial activity.

### Study Overview

In the examples illustrated below, the antibacterial activities of a variety of inventive and comparative compositions were measured. Comparison studies below illustrate that addition of aluminum compounds and transitional metal compounds to kaolinic clays leads to a profound increase in antibacterial activity of the resulting compositions. The data below illustrates that the antibacterial activity of compositions of the present disclosure can be modulated is dose-dependent manner-based on the proportion of the aluminum compound, the transitional metal compound, or both, relative to the proportion of the kaolinic clay.

### Materials

Commercial kaolin was obtained from obtained from IMERYS Kaolin. Commercial aluminum (III) sulfate [Al₂(SO₄)₃.14H₂O] in the form of Liquid Alum (48.5 wt. %) was obtained from General Chemistry Corporation. Commercial iron (II) sulfate heptahydrate [FeSO₄.7H₂O] (>99% by weight purity) was obtain from Fisher Scientific. Commercial copper (II) sulfate pentahydrate [CuSO₄.5H₂O] (>99% by weight purity) was obtain from LabChem, Inc. Commercial calcium bentonite clay (Bavarian) was obtained from Imerys Performance Minerals.

Preparation of Kaolin Samples:

Crudes samples of grey, pyritic kaolin were combined, crushed, and then placed on a glass baking sheet and dried at about 105° C. for about 12 hours. The resulting dried sample was then pulverized using a micropulverizer fitted with a #02 screen (1 pass) to form a dry-sized kaolin sample.

Chemical analysis was performed on two different kaolin clay samples (Kaolin #1 and Kaolin #2) using X-ray fluorescence spectrometry including total sulfur assay with Horiba EMIA-320V2 Analyzer. The chemical analyses of Kaolin #1 and Kaolin #2 are shown in Table 1 below.

As illustrated in Table 1 below, the content of pyrite (FeS₂) in kaolin clays can vary over a sizeable range—in this case from a relatively small amount of 0.05 wt. % in Kaolin #1 to a significantly higher amount of 0.28 wt. % in Kaolin #2. Embodiments of the present disclosure can greatly increase the antibacterial activity of kaolinic clays such as Kaolin #1 which, due to its low content of pyrite, exhibits a relatively high oxidation-reduction potential (ORP) and therefore low activity towards bacteria such as E. coli.

General Preparation of Antibacterial Compositions:

Experimental compositions were generally prepared using a dry-sized kaolin sample, i.e., the Kaolin #1 described above. Aqueous kaolin samples were prepared at 50% solids content using filtered, deionized water. Treated clay samples were then produced by adding at least one metal-containing compound (e.g., aluminum compound and/or transition metal compound) followed by thorough mixing to form dispersions, which were then dried at about 105° C. for 12 hours and pulverized.

General Preparation of E. Coli Samples:

E. coli were grown in a sterile growth medium (i.e., Luria Broth (LB)) for about 24 hours—at which time the initial cultures were counted and found to have a level of growth of 7.8 log₁₀ CFU/ml (colony forming units per ml).

TABLE 1
Chemical Analysis of Kaolin Clays

| Sample ID | Kaolin # 1 | Kaolin # 2 |
|---|---|---|
| Fe₂O₃(wt. %) | 0.82 | 1.03 |
| MgO(wt. %) | 0.10 | 0.04 |
| Al₂O₃(wt. %) | 38.99 | 38.41 |
| SiO₂(wt. %) | 43.71 | 42.75 |
| TiO₂(wt. %) | 1.50 | 2.74 |
| CaO(wt. %) | 0.06 | 0.03 |
| Na₂O(wt. %) | 0.15 | 0.04 |
| K₂O(wt. %) | 0.34 | 0.06 |
| P₂O₅(wt. %) | 0.10 | 0.09 |
| Horiba S(wt. %) | 0.03 | 0.15 |
| Loss on ignition(wt. %) | 14.65 | 14.65 |
| Total(wt. %) | 100.0 | 100.0 |
| Pyrite(wt. %) (calculated | 0.05 | 0.28 |
| from Sulfur) | | |
| Aqueous Soluble Al Cations | 166 mg/Kg | 240 |
| (determined by ICP/AES) | | |
| Aqueous Soluble Fe Cations | 195 mg/Kg | 180 |
| (determined by ICP/AES) | | |
| pH in water (at 20% solids | n/m | 3.40 |
| content) | | |
| ORP in water (at 20% solids | n/m | 435 |
| content) | | |

n/m not measured

General Preparation of Test Samples:

2.5 grams of control clay or a treated clay were then added to a sterile centrifuge tube, and the volume in the tube is brought to 12.5 ml using sterile water and then up to 25 ml (total) using a sterile LB broth to form an initial broth sample. Then 1 ml of the initial culture of E. coli (7.8 log₁₀ CFU/ml) was added to the initial broth sample, and the resulting sample was incubated at 37° C. and shaken at 85 rpms for a period of time ranging from 24 to 48 hours. Controls for each sample were also prepared by adding 2.5 grams of control clay or treated clay to a sterile centrifuge tube, and the volume in the tube was brought to 12.5 ml using sterile water and then up to 25 ml (total) using the sterile LB broth without any E. coli being added. The control samples were then incubated at 37° C. and shaken at 85 rpms for the period ranging from 24 to 48 hours.

Sample Incubation and Measurements:

After each sample set (E. coli inoculated and non-inoculated control) was incubated for the desired time period (24 to 48 hours), and the sample set was allowed to settle without agitation till the sample set distinguished itself into a solids layer and a liquid layer. Each sample set was evaluated using at least one of the following assays: (1) a CFU-based bacterial assay; (2) a UV-visible bacterial assay; (3) a pH measurement; and (4) an oxidation-reduction potential (ORP) measurement.

CFU-Based Bacterial Assay:

As explained above, following sample incubation the samples were evaluated for the level of bacterial growth using a CFU-based bacterial assay and/or a UV-visible bacterial assay. The CFU-based bacterial assay involves serially diluting samples by various dilution factors, plating the surface of pre-poured LB plates, and manually counting the resulting sample cultures to measure the level of growth based on log₁₀ CFU/ml. CFU per ml were calculated by counting the number of colonies on the plate with growths ranging from 30 and 300, and then applying the equation (1):

$$\frac{CFU}{ml} - \frac{( \# of colonies ) \times ( dilution factor )}{( volume plated )} . \qquad ( 1 )$$

In this CFU-based bacterial assay, the levels of activity corresponding to the control samples (no E. coli) were set as the zero points.

UV-Visible Bacterial Assay:

The UV-visible bacterial assay involves measuring the level of UV-visible absorbance of the samples at a wavelength of 570 nm in order to determine the magnitudes of optical density (OD) for the samples. As explained below in greater detail, the magnitudes of optical density (OD) for the samples were used as an indication of bacterial viability and to approximate the CFU per ml of the sample. When measuring the optical density (OD) of a sample, the level of activity corresponding to the control samples (no E. coli) were set as the zero points.

Oxidation-Reduction Potential (ORP) Measurements:

ORP measurements of the experimental samples were carried out using an Oakton Optester® 10 instrument following conditioning of the electrode by immersion in electrode storage solution or tap water for at least 30 minutes. Readouts were taken in millivolts (mV).

### Binary Compositions: The Effects on Antibacterial Activity of Separately Adding Iron (II) Sulfate and Aluminum (III) Sulfate to a Kaolin Clay

A control E. coli sample designated as Example 1 was prepared as described in the general preparation procedure above. A control sample of the Kaolin #1 clay designated as Example 2 was prepared as a water dispersion having a 50% solids content.

A binary composition designated as Example 3 was prepared as a water dispersion containing 0.31 wt. % of iron sulfate relative to a total weight of the Kaolin #1 clay having a 50% solids content. A binary composition designated as Example 4 was prepared as a water dispersion containing 0.62 wt. % of iron sulfate relative to a total weight of the Kaolin #1 clay having a 50% solids content. A binary composition designated as Example 5 was prepared as a water dispersion containing 1.24 wt. % of iron sulfate relative to a total weight of the Kaolin #1 clay having a 50% solids content. A binary composition designated as Example 6 was prepared as a water dispersion containing 2.48 wt. % of iron sulfate relative to a total weight of the Kaolin #1 clay having a 50% solids content. A binary composition designated as Example 7 was prepared as a water dispersion containing 1.24 wt. % of aluminum sulfate (Alum) relative to a total weight of the Kaolin #1 clay having a 50% solids content. A binary composition designated as Example 8 was prepared as a water dispersion containing 2.48 wt. % of aluminum sulfate (Alum) relative to a total weight of the Kaolin #1 clay having a 50% solids content.

Examples 1-8 were prepared according to the general preparation of test samples described above, and were then subjected to the sample incubation and measurements described above. Table 2 below summarizes the experimental results for Examples 1-8 in which the samples were measured using the direct bacterial assay for CFU concentration after periods of 24 and 48 hours, and both pH and ORP were measured at 48 hours.

TABLE 2
Effect of Metal-Containing Compounds on ABC Activity of Kaolin

| | Example | | 24 Hour | 48 Hour | 48 Hour | 48 Hour |
|---|---|---|---|---|---|---|
| Ex. # | Contents | Amounts | log₁₀ CFU/ml | log₁₀ CFU/ml | pH | ORP |
| 1 | E. Coli | 100 wt. % | 7.8 | 7.9 | 8.05 | 250 mV |
| | (control) | | | | | |
| 2 | Kaolin # 1 | 100 wt. %^{a)} | 3.1 | 5.1 | 4.78 | 435 mV |
| 3 | FeSO₄•7H₂O | 0.31 wt. %^{b)} | 3.6 | 3.5 | 4.52 | 598 mV |
| | Kaolin # 1 | remainder^{a)} | | | | |
| 4 | FeSO₄•7H₂O | 0.62 wt. %^{c)} | 3.4 | 0 | 4.33 | 603 mV |
| | Kaolin # 1 | remainder^{a)} | | | | |
| 5 | FeSO₄•7H₂O | 1.24 wt. %^{d)} | 2.9 | 0 | 4.11 | 623 mV |
| | Kaolin # 1 | remainder^{a)} | | | | |
| 6 | FeSO₄•7H₂O | 2.48 wt. %^{e)} | 0 | 0 | 3.81 | 686 mV |
| | Kaolin # 1 | remainder^{a)} | | | | |
| 7 | Al₂(SO₄)₃•14H₂O | 1.24 wt. %^{f)} | 0 | 0 | 3.91 | 485 mV |
| | Kaolin # 1 | remainder^{a)} | | | | |
| 8 | Al₂(SO₄)₃•14H₂O | 2.48 wt. %^{e)} | 0 | 0 | 3.56 | 530 mV |
| | Kaolin # 1 | remainder^{a)} | | | | |

^{a)}50% solids content of Kaolin #1 in water
^{b)}6.25 lbs of compound per ton of clay
^{c)}12.5 lbs of compound per ton of clay
^{d)}25 lbs of compound per ton of clay
^{e)}50 lbs of compound per ton of clay

As shown in Table 2 above, the control samples of Examples 1 and 2 exhibited high bacterial activities after 24 and 48 hours. The Kaolin #1 clay of Example 2 exhibited some initial antibacterial activity as indicated by the reduction in CFU concentration after 24 hours. However, after an additional period of 24 hours (48 hours total), the bacterial activity of Example 2 had increased from 3.1 (24 hours) to 5.1 (48 hours)—indicating a saturation of the antibacterial activity of the Kaolin #1 clay after a period of 24 hours.

As illustrated in Examples 3-8 in Table 2 above, the binary samples containing iron sulfate exhibited antibacterial activity in a dose-dependent manner. Although the iron sulfate-containing sample of Example 3 exhibited significant bacterial activity after a period of 48 hours, increasing the proportion of iron sulfate from 0.31 wt. % (Example 3) to 0.62 wt. % (Example 4) resulted in no measurable CFU concentration after a period of 48 hours. This trend continued as the proportion of iron sulfate was further increased in Example 5 (1.24 wt. %) and Example 6 (2.48 wt. %). The iron sulfate-containing sample of Example 6 (2.48 wt. %) exhibited no measurable CFU concentration after periods of 24 and 48 hours.

As illustrated in Examples 7 and 8 in Table 2 above, the binary samples containing aluminum sulfate also exhibited antibacterial activity in a dose-dependent manner. Unlike the iron sulfate-containing sample of Example 5 (1.24 wt. %), which exhibited bacterial activity at 24 hours, the aluminum sulfate-containing sample of Example 7 (1.24 wt. %) was free of measurable CFU concentration at both 24 and 48 hours. The aluminum sulfate-containing sample of Example 8 (2.48 wt. %) exhibited no measurable bacterial activity at 24 or 48 hours. The data in Table 2 suggests that aluminum sulfate is more potent than iron sulfate—in terms of enhancing the antibacterial activity of the Kaolin #1 clay.

As shown in Table 2 above, the addition of increasing amount iron sulfate and aluminum sulfate resulted in dose-dependent decreases in the 48 hour pH of Examples 3-8, relative to the pH of the control Example 2. As shown in Table 2 above, the addition of increasing amount of iron sulfate and aluminum sulfate also resulted in dose-dependent increases in the 48 hour oxidation-reduction potential (ORP) of Examples 3-8, relative to the ORP of the control Example 2.

### Binary Compositions: The Effects on Antibacterial Activity of Separately Adding a Bentonite Clay, Iron (II) Sulfate, Copper (II) Sulfate and Aluminum (III) Sulfate to a Kaolin Clay

A control E. coli sample designated as Example 9 was prepared as described in the general preparation above. A control sample of the Kaolin #1 clay designated as Example 10 was prepared as a water dispersion having a 50% solids content.

A binary composition designated as Example 11 was prepared as a water dispersion containing 25 wt. % of bentonite clay and 75 wt. % of the Kaolin #1 having a 50% solids content. A binary composition designated as Example 12 was prepared as a water dispersion containing 50 wt. % of bentonite clay and 50 wt. % of the Kaolin #1 having a 50% solids content.

A binary composition designated as Example 13 was prepared as a water dispersion containing 1.24 wt. % of iron sulfate relative to a total weight of the Kaolin #1 clay having a 50% solids content. A binary composition designated as Example 14 was prepared as a water dispersion containing 2.48 wt. % of iron sulfate relative to a total weight of the Kaolin #1 clay having a 50% solids content. A binary composition designated as Example 15 was prepared as a water dispersion containing 2.48 wt. % of copper sulfate relative to a total weight of the Kaolin #1 clay having a 50% solids content. A binary composition designated as Example 16 was prepared as a water dispersion containing 1.24 wt. % of aluminum sulfate (Alum) relative to a total weight of the Kaolin #1 clay having a 50% solids content. A binary composition designated as Example 17 was prepared as a water dispersion containing 1.85 wt. % of aluminum sulfate (Alum) relative to a total weight of the Kaolin #1 clay having a 50% solids content. A binary composition designated as Example 18 was prepared as a water dispersion containing 2.48 wt. % of aluminum sulfate (Alum) relative to a total weight of the Kaolin #1 clay having a 50% solids content.

Examples 9-18 were prepared according to the general preparation of test samples described above, and were then subjected to the sample incubation and measurements described above. Table 3 below summarizes the experimental results for Examples 9-18 in which the samples were measured using the direct bacterial assay for CFU concentration after a period of 24 hours.

TABLE 3
Effect of Bentonite Clay and Metal-Containing
Compounds on ABC Activity of Kaolin

| Exam- | | | | 24 Hour |
|---|---|---|---|---|
| ple No. | Example Contents | Amounts | | log₁₀ CFU/ml |
| 9 | E. Coli (control) | 100 | wt. % | 7.8 |
| 10 | Kaolin # 1 | 100 | wt. % ^{a)} | 7.6 |
| 11 | Bentonite | 25 | wt. % | 7.5 |
| | Kaolin # 1 | remainder ^{a)} | | |
| 12 | Bentonite | 50 | wt. % | 7.5 |
| | Kaolin #1 | remainder ^{a)} | | |
| 13 | FeSO₄•7H₂O | 1.24 | wt. % ^{d)} | 6.8 |
| | Kaolin # 1 | remainder ^{a)} | | |
| 14 | FeSO₄•7H₂O | 2.48 | wt. % ^{e)} | 0 |
| | Kaolin # 1 | remainder ^{a)} | | |
| 15 | CuSO₄•5 H₂O | 2.48 | wt. % ^{e)} | 0 |
| | Kaolin # 1 | remainder ^{a)} | | |
| 16 | Al₂(SO₄)₃•14 H₂O | 1.24 | wt. % ^{d)} | 0 |
| | Kaolin # 1 | remainder ^{a)} | | |
| 17 | Al₂(SO₄)₃•14 H₂O | 1.86 | wt. % ^{f)} | 0 |
| | Kaolin # 1 | remainder ^{a)} | | |
| 18 | Al₂(SO₄)₃•14 H₂O | 2.48 | wt. % ^{e)} | 0 |

| | |
|---|---|
| Kaolin # 1 | remainder ^{a)} |

^{a)} 50% solids content of Kaolin #1 in water
^{d)} 25 lbs of compound per ton of clay
^{e)} 50 lbs of compound per ton of clay
^{f)} 37.5 lbs of compound per ton of clay

As shown in Table 3 above, the control samples of Examples 9 and 10 exhibited high bacterial activities after 24 hours. The Kaolin #1 clay of Example 10 exhibited some initial antibacterial activity as indicated by the slight reduction in CFU concentration after 24 hours.

As illustrated in Examples 11 and 12 in Table 3 above, the binary samples containing bentonite exhibited almost no increase in antibacterial activity compared to the Kaolin #1 control of Example 10. The bentonite-containing sample of Example 11 (25 wt. %) exhibited only a slight decrease in the CFU concentration relative to the Kaolin #1 control of Example 10. Increasing the proportion of bentonite to 50 wt. % in Example 12 resulting in no decrease in the CFU concentration relative to the bentonite-containing sample of Example 11. Therefore, the presence of bentonite does not appear to increase the anti-bacterial activity of the Kaolin #1 clay.

As illustrated in Examples 13-14 in Table 3 above, the binary samples containing iron sulfate exhibited antibacterial activity in a dose-dependent manner. Although the iron sulfate-containing sample of Example 13 exhibited significant bacterial activity (log₀ CFU/ml=6.8) after a period of 24 hours, increasing the proportion of iron sulfate from 1.24 wt. % (Example 13) to 2.48 wt. % (Example 14) resulted in no measurable CFU concentration after a period of 24 hours.

As illustrated in Example 15 in Table 3 above, the binary sample containing 2.48 wt. % of copper sulfate exhibited no measurable CFU concentration after a period of 24 hours. Therefore, it appears that copper sulfate is also effective in increasing the antibacterial activity of the Kaolin #1 clay.

As illustrated in Examples 16-18 in Table 3 above, the binary samples containing aluminum sulfate also exhibited antibacterial activity. Unlike the iron sulfate-containing sample of Example 13 (1.24 wt. %), which exhibited bacterial activity at 24 hours, the aluminum sulfate-containing sample of Example 16 (1.24 wt. %) was free of measurable CFU concentration at 24. As expected, the aluminum sulfate-containing samples of Examples 17 and 18 (1.86 wt. % and 2.48 wt. %) also exhibited no measurable bacterial activity after 24 hours. The data in Table 3 also suggests that aluminum sulfate is more potent than iron sulfate—in terms of enhancing the antibacterial activity of the Kaolin #1 clay.

### The Antibacterial Activities of Iron (II) Sulfate, Copper (II) Sulfate and Aluminum (III) Sulfate in the Absence of a Kaolin Clay

A control E. coli sample designated as Example 19 was prepared as described in the general preparation above. A number of control examples were also prepared that omitted the Kaolin #1 clay.

A control sample designated as Example 20 was prepared as a water dispersion containing the same amount of iron sulfate that was used in Example 4—but omitting the Kaolin #1 clay. The proportion of iron sulfate in Example 20 was thereby normalized to emulate the amount of 0.62 wt. % used in Example 4. A control sample designated as Example 21 was prepared as a water dispersion containing the same amount of iron sulfate that was used in Examples 6—but omitting the Kaolin #1 clay. The proportion of iron sulfate in Example 21 was thereby normalized to emulate the amount of 2.48 wt. % used in Example 6.

A control sample designated as Example 22 was prepared as a water dispersion containing the same amount of copper sulfate as the amount of iron sulfate that was used in Example 3—but omitting the Kaolin #1 clay. The proportion of copper sulfate in Example 22 was thereby normalized to emulate the amount of 0.31 wt. % used in Example 3. A control sample designated as Example 23 was prepared as a water dispersion containing the same amount of copper sulfate as the amount of iron sulfate that was used in Example 4—but omitting the Kaolin #1 clay. The proportion of copper sulfate in Example 23 was thereby normalized to emulate the amount of 0.62 wt. % used in Example 4. A control sample designated as Example 24 was prepared as a water dispersion containing the same amount of copper sulfate as the amount of iron sulfate that was used in Example 6—but omitting the Kaolin #1 clay. The proportion of copper sulfate in Example 24 was thereby normalized to emulate the amount of 2.48 wt. % used in Example 6.

A control sample designated as Example 25 was prepared as a water dispersion containing the same amount of aluminum sulfate (Alum) as the amount of iron sulfate that was used in Example 4—but omitting the Kaolin # 1 clay. The proportion of iron sulfate in Example 25 was thereby normalized to emulate the amount of 0.62 wt. % used in Example 4. A control sample designated as Example 26 was prepared as a water dispersion containing the same amount of aluminum sulfate (Alum) that was used in Examples 8—but omitting the Kaolin #1 clay. The proportion of aluminum sulfate in Example 26 was thereby normalized to emulate the amount of 2.48 wt. % used in Example 8.

Examples 19-26 were prepared according to the general preparation of test samples described above, and were then subjected to the sample incubation and measurements described above. Table 4 below summarizes the experimental results for Examples 19-26 in which the samples were measured using the bacterial assay for CFU concentration after a period of 24 hours. Based on preliminary observations of a correlation between optical properties and CFU levels, the optical density (OD) (absorbance) at 570 nm also was measured by UV-visible spectroscopy after a period of 24 hours as an indication of bacterial activity.

As shown in Table 4 below, the control sample of Examples 19 exhibited a high bacterial activity after 24 hours, based on the high CFU concentration (log 10 CFU/ml=9.0) and high optical density (UV-visible absorption of 1.389 at 570 nm). As shown in Table 4 above, the control samples of Examples 20-26 exhibited dose-dependent antibacterial activities, indicating that iron sulfate, copper sulfate and aluminum sulfate can all reduce the bacterial activity of E. coli in the absence of the Kaolin #1 clay.

TABLE 4
ABC Activity of Metal-Containing Compounds without Clay

| | | | | 24 Hour | UV-Vis |
|---|---|---|---|---|---|
| Exam- | | | | log₁₀ | Abs @ |
| ple No. | Example Contents | Amounts | | CFU/ml | 570 nm ^{g)} |
| 19 | E. Coli (control) | 100 | wt. % | 9.0 | 1.389 ^{s)} |
| 20 | FeSO₄•7H₂O | 0.62 | wt. % ^{h)} | 8.8 | 1.172 ^{s)} |
| 21 | FeSO₄•7H₂O | 2.48 | wt. % ⁱ⁾ | 0 | 0.291 ^{q)} |
| 22 | CuSO₄•5 H₂O | 0.31 | wt. % ^{j)} | 7.8 | 0.734 ^{r)} |
| 23 | CuSO₄•5 H₂O | 0.62 | wt. % ^{k)} | 2.4 | ^{t)} |
| 24 | CuSO₄•5 H₂O | 2.48 | wt. % ^{l)} | 0 | 0.055 ^{p)} |
| 25 | Al₂(SO₄)₃•14 H₂O | 0.62 | wt. % ^{m)} | 8.5 | 1.377 ^{s)} |
| 26 | Al₂(SO₄)₃•14 H₂O | 2.48 | wt. % ⁿ⁾ | 0 | ^{u)} |

^{g)} Optical Density (OD)
^{h)} No Kaolin clay present - proportion normalized to the same amount used in Example 4
ⁱ⁾ No Kaolin clay present - proportion normalized to the same amount used in Example 6
^{j)} No Kaolin clay present - proportion normalized to the same amount used in Example 3
^{k)} No Kaolin clay present - proportion normalized to the same amount used in Example 4
^{l)} No Kaolin clay present - proportion normalized to the same amount used in Example 6
^{m)} No Kaolin clay present - proportion normalized to the same amount used in Example 4
ⁿ⁾ No Kaolin clay present - proportion normalized to the same amount used in Example 8
^{o)} Complete Kill Candidate (<0.250)
^{p)} Inhibition-Kill Candidate (0.250-0.350)
^{q)} Inhibition Candidate (0.351-0.450)
^{r)} <10⁸ Growth Candidate (0.451-1.250)
^{s)} Robust Growth Candidate (>1.251)
^{t)} Sediment disturbance of the control sample prohibited accurate optical density (OD) measurement.
^{u)} Sediment disturbance of the experimental sample prohibited accurate optical density (OD) measurement.

As illustrated in Example 20 of Table 4 above, a sample containing 0.62 wt. % of iron sulfate causes only a slight reduction in bacterial activity after a period of 24 hours. However, increasing the proportion of the iron sulfate to 2.48 wt. % in the sample of Example 21 resulted in no measurable CFU concentration after 24 hours, and a significant reduction in the optical density from 1.172 (Example 20) to 0.291 (Example 21).

As illustrated in Example 25 of Table 4 above, a sample containing 0.62 wt. % of aluminum sulfate causes only a slight reduction in bacterial activity after a period of 24 hours. However, increasing the proportion of the aluminum sulfate to 2.48 wt. % in the sample of Example 26 resulted in no measurable CFU concentration after 24 hours, and a significant reduction in the optical density from 1.377 (Example 25) to 0.812 (Example 26).

Indirect Bacterial Assay:

Due to the cumbersome nature of the CFU-based bacterial assay described above, an indirect bacterial assay has also been developed which uses the optical density (OD) of a sample measured by UV-visible spectroscopy to approximate the CFU per ml of the sample. The Examples illustrated in Table 4 demonstrate a significant correlation between the CFU concentration after 24 hours and the optical density (OD) of a sample, thus indicating the optical density (OD) of a sample can also be used as an indirect measure to evaluate the bacterial viability of the sample-thereby evaluating the antibacterial potential of the sample.

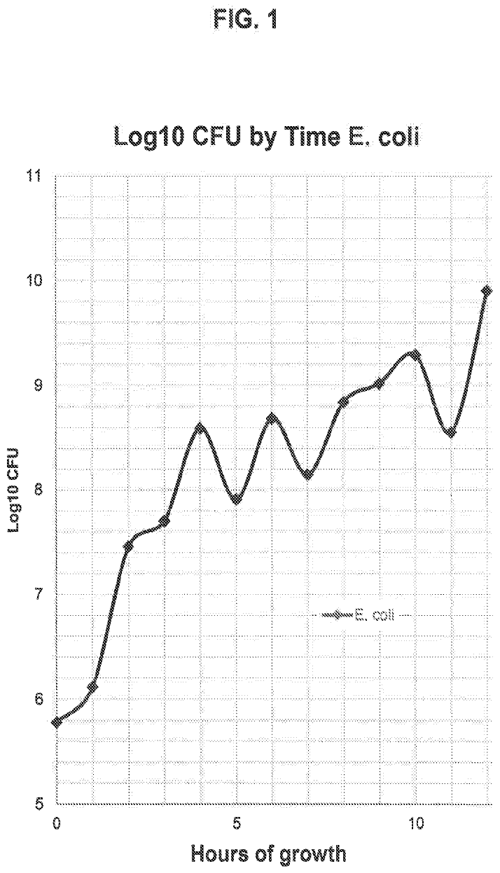

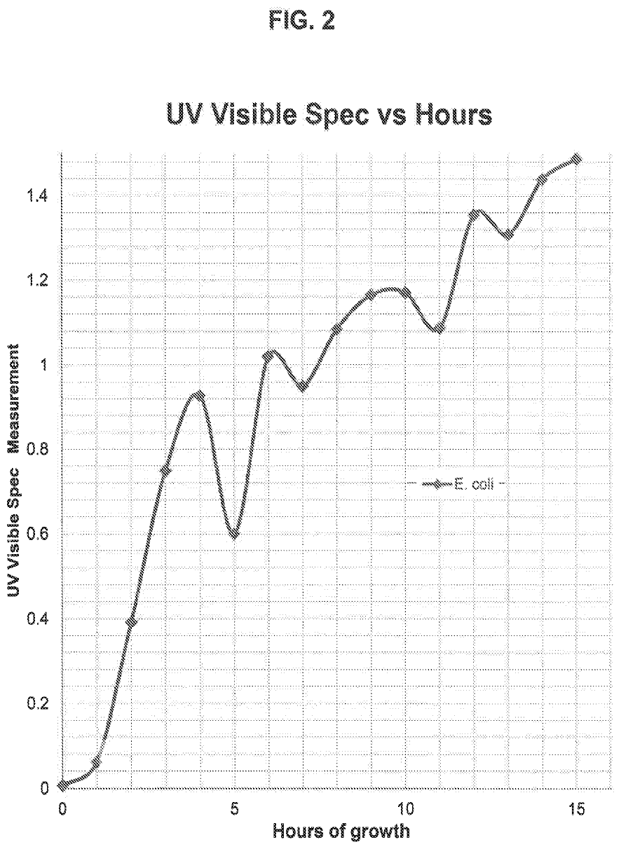

As illustrated in FIGS. 1 and 2, the growth of E. coli as measured by the CFU-based bacterial assay and the UV-visible bacterial assay follows a similar trend over time periods ranging from about 12 to 15 hours. From this observation it was realized that an indirect bacterial assay could be devised which uses the optical density (OD) of a sample measured in the UV-visible bacterial assay to approximate the CFU concentration of the sample.

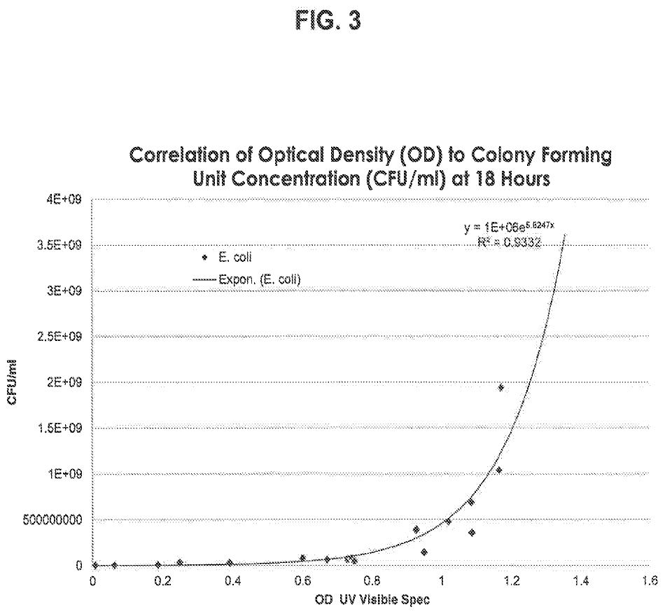

In order to relate the optical density (OD) of the samples to an estimate CFU concentration, 18 E. coli samples and a non-inoculated control sample were incubated for 1-18 hours. Each hour the corresponding samples were subjected to serial dilution, plated, and CFUs determined by colony growth to produce the correlation chart shown in FIG. 3. Evaluation of data determined an initial OD reading after inoculation to be +0.250 (i.e., after inoculation, and before a complete kill some time elapsed allowing for initial growth, this growth corresponded to up to 0.250, and these samples after 24 hours did not have living colonies when plated despite a positive OD number). FIG. 3 is a graph showing the correlation of optical density (OD) to CFU concentration for the E. coli sample. It was found that the CFU concentration of an E. coli sample is exponentially related to the OD of the sample at an inoculation period of 18 hours. Therefore, using the OD measured in the UV-visible bacterial assay described above, the CFU concentration may be approximated using equation (2):

$$\frac{CFU}{ml} = 1 \times {10}^{6} ( {e}^{5.825 \times OD} ) . \qquad ( 2 )$$

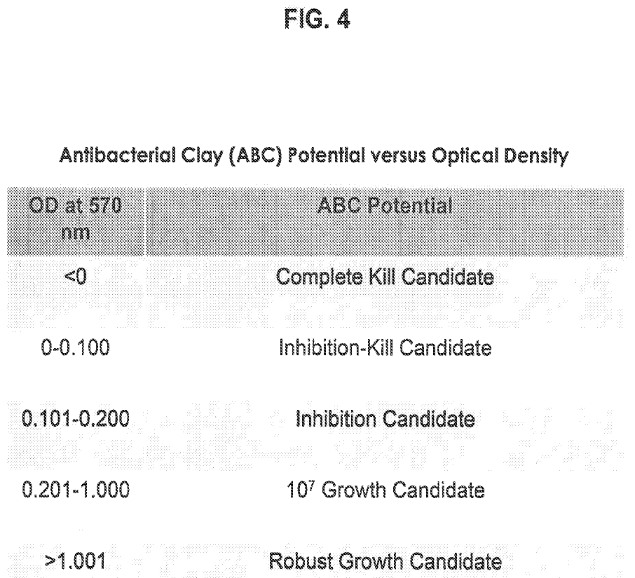

OD Evaluation of Antibacterial Potential: The optical density (OD) of a sample can also be used as an indirect measure to evaluate the antibacterial potential of a sample, FIG. 4 is a chart of antibacterial clay (ABC) potential versus optical density (OD) at 570 nm. Under this methodology, the normalized OD value is used to categorize the sample's antibacterial potential-whereby an optical density (OD) of less than 0 indicates a “Complete Kill Candidate”; an optical density (OD) ranging from 0 to 0.100 indicates an “Inhibition-Kill Candidate”; an optical density (OD) ranging from 0.101 to 0.200 indicates an “Inhibition Candidate”; an optical density (OD) ranging from 0.201 to 1.000 indicates a “10⁷ Growth Candidate”; and an optical density (OD) of greater than 1.000 indicates a “Robust Growth Candidate,” The normalized OD values in Table 5 correspond to the measured OD values minus 0.250 (to account for the short growth period after initial inoculation discussed above).

### Ternary Compositions: The Effect on Antibacterial Activity of Adding Iron (II) Sulfate and Aluminum (III) Sulfate to a Kaolin Clay

A control E. coli sample designated as Example 27 was prepared as described in the general preparation above. A control sample of the Kaolin #1 clay designated as Example 28 was prepared as a water dispersion having a 50% solids content.

A ternary composition designated as Example 29 was prepared as a water dispersion containing 0.31 wt. % of aluminum sulfate (Alum) and 0.31 wt. % of iron sulfate relative to a total weight of the Kaolin #1 clay having a 50% solids content. A ternary composition designated as Example 30 was prepared as a water dispersion containing 0.31 wt. % of aluminum sulfate (Alum) and 0.62 wt. % of iron sulfate relative to a total weight of the Kaolin #1 clay having a 50% solids content. A ternary composition designated as Example 31 was prepared as a water dispersion containing 0.31 wt. % of aluminum sulfate (Alum) and 1.24 wt. % of iron sulfate relative to a total weight of the Kaolin #1 clay having a 50% solids content. A ternary composition designated as Example 32 was prepared as a water dispersion containing 0.62 wt. % of aluminum sulfate (Alum) and 0.31 wt. % of iron sulfate relative to a total weight of the Kaolin #1 clay having a 50% solids content. A ternary composition designated as Example 33 was prepared as a water dispersion containing 0.62 wt. % of aluminum sulfate (Alum) and 0.62 wt. % of copper sulfate relative to a total weight of the Kaolin #1 clay having a 50% solids content. A ternary composition designated as Example 34 was prepared as a water dispersion containing 0.62 wt. % of aluminum sulfate (Alum) and 1.24 wt. % of copper sulfate relative to a total weight of the Kaolin #1 clay having a 50% solids content. A ternary composition designated as Example 35 was prepared as a water dispersion containing 1.24 wt. % of aluminum sulfate (Alum) and 0.31 wt. % of copper sulfate relative to a total weight of the Kaolin #1 clay having a 50% solids content. A ternary composition designated as Example 36 was prepared as a water dispersion containing 1.24 wt. % of aluminum sulfate (Alum) and 0.62 wt. % of copper sulfate relative to a total weight of the Kaolin #1 clay having a 50% solids content. A ternary composition designated as Example 37 was prepared as a water dispersion containing 1.24 wt. % of aluminum sulfate (Alum) and 1.24 wt. % of copper sulfate relative to a total weight of the Kaolin #1 clay having a 50% solids content.

Examples 27-37 were prepared according to the general preparation of test samples described above, and were then subjected to the sample incubation and measurements described above. Table 5 below summarizes the experimental results for Examples 27-37 in which the samples were measured using the direct bacterial assay for optical density (OD) after a period of 48 hours, and both pH and ORP were measured at 48 hours.

TABLE 5
Effect of Metal-Containing Compounds on ABC Activity of Kaolin

| | | | | | | 48 |
|---|---|---|---|---|---|---|
| | | | | UV-Vis | 48 | Hour |
| Exam- | Example | | | Abs @ | Hour | ORP |
| ple No. | Contents | Amounts | | 570 nm^{u)} | pH | (mV) |
| 27 | E. Coli (control) | 100 | wt. % | 0.993^{y)} | 6.90 | 250 |
| 28 | Kaolin # 1 | 100 | wt. % ^{a)} | 0.236^{x)} | 3.40 | 435 |
| 29 | Al₂(SO₄)₃•14 H₂O | 0.31 | wt. % ^{b)} | −0.048^{v)} | 2.88 | 426 |
| | FeSO₄•7H₂O | 0.31 | wt. % ^{b)} | | | |
| | Kaolin # 1 | remainder ^{a)} | | | | |
| 30 | Al₂(SO₄)₃•14 H₂O | 0.31 | wt. % ^{b)} | −0.150^{v)} | 2.82 | 425 |
| | FeSO₄•7H₂O | 0.62 | wt. % ^{c)} | | | |
| | Kaolin # 1 | remainder ^{a)} | | | | |
| 31 | Al₂(SO₄)₃•14 H₂O | 0.31 | wt. % ^{b)} | −0.042^{v)} | 2.76 | 432 |
| | FeSO₄•7H₂O | 1.24 | wt. % ^{d)} | | | |
| | Kaolin # 1 | remainder ^{a)} | | | | |
| 32 | Al₂(SO₄)₃•14 H₂O | 0.62 | wt. % ^{c)} | −0.093^{v)} | 2.85 | 434 |
| | FeSO₄•7H₂O | 0.31 | wt. % ^{b)} | | | |
| | Kaolin # 1 | remainder ^{a)} | | | | |
| 33 | Al₂(SO₄)₃•14 H₂O | 0.62 | wt. % ^{c)} | −0.115^{v)} | 2.84 | 430 |
| | FeSO₄•7H₂O | 0.62 | wt. % ^{c)} | | | |
| | Kaolin # 1 | remainder ^{a)} | | | | |
| 34 | Al₂(SO₄)₃•14 H₂O | 0.62 | wt. % ^{c)} | −0.129^{v)} | 2.77 | 441 |
| | FeSO₄•7H₂O | 1.24 | wt. % ^{d)} | | | |
| | Kaolin #1 | remainder ^{a)} | | | | |
| 35 | Al₂(SO₄)₃•14 H₂O | 1.24 | wt. % ^{d)} | −0.200^{v)} | 2.92 | 424 |
| | FeSO₄•7H₂O | 0.31 | wt. % ^{b)} | | | |
| | Kaolin # 1 | remainder ^{a)} | | | | |
| 36 | Al₂(SO₄)₃•14 H₂O | 1.24 | wt. % ^{d)} | −0.237^{v)} | 2.80 | 420 |
| | FeSO₄•7H₂O | 0.62 | wt. % ^{c)} | | | |
| | Kaolin # 1 | remainder ^{a)} | | | | |
| 37 | Al₂(SO₄)₃•14 H₂O | 1.24 | wt. % ^{d)} | −0.173^{v)} | 2.78 | 426 |
| | FeSO₄•7H₂O | 1.24 | wt. % ^{d)} | | | |

| | |
|---|---|
| Kaolin # 1 | remainder ^{a)} |

^{a)} 50% solids content of Kaolin #1 in water
^{b)} 6.25 lbs of compound per ton of day
^{c)} 12.5 lbs of compound per ton of clay
^{d)} 25 lbs of compound per ton of clay
^{u)}Optical Density (OD), normalized by subtracting 0.250 from measured UV absorptions
^{v)}Normalized Complete Kill Candidate (<0)
^{w)} Normalized Inhibition-Kill Candidate (0-0.200)
^{x)}Normalized Inhibition Candidate (0.201-0.400)
^{y)}Normalized <10⁸ Growth Candidate (0.401-1.000)
^{z)} Normalized Robust Growth Candidate (>1.001)

As shown in Table 5 above, the control sample of Example 27 exhibited a high bacterial activity after 48 hours. The Kaolin #1 clay control sample of Example 28 exhibited a significantly lower bacterial activity as evidenced by the reduction in optical density of Example 28 (0.236) versus the optical density of the E. coli control sample of Example 27 (0.993).

As shown in Table 5 above, the ternary composition of Example 29 resulted in an optical density of −0.048. Therefore, the ternary composition of Example 29 does not contain enough of the aluminum sulfate and/or the iron sulfate to sufficiently lower the bacterial activity of E. coli.

As shown in Table 5 above, the ternary compositions of Examples 32-34 resulted in optical densities decreasing from −0.093 to −0.129. Therefore, the ternary compositions of Examples 32-34 contain enough of the aluminum sulfate and/or the iron sulfate to inhibit the bacterial activity of E. co/i, but not kill the E. coli.

As shown in Table 5 above, the ternary compositions of Examples 30 and 35-37 resulted in optical densities of −0.150, −0.200, −0.237 and −0.173. Therefore, the ternary compositions of Examples 30 and 35-37 contain enough of the aluminum sulfate and/or the iron sulfate to inhibit the bacterial activity of E. coli, and to kill at least some of the E. coli.

The experimental data in Table 5 above provides additional evidence suggesting that the aluminum sulfate (Alum) is more potent than the iron sulfate. Comparing, for example, the results for the samples of Examples 30 and 32 shows that a ternary composition containing 0.31 wt. % of aluminum sulfate and 0.62 wt. % of iron sulfate exhibits a lower optical density of 0.100 (Example 30) compared to the optical density of −0.093 (Example 32) for a corresponding ternary composition containing 0.62 wt. % of aluminum sulfate and 0.31 wt. % of iron sulfate.

The above description is presented to enable a person skilled in the art to make and use the invention. Certain aspects are provided in the context of a particular application and its requirements for illustration purposes. Various modifications to the embodiments disclosed herein will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the disclosure may not show every benefit of the invention, considered broadly.

1. An antimicrobial composition, comprising:
a clay; and
an aluminum compound,
wherein:
a pH of the antimicrobial composition is less than or equal to 5; and
an oxidation-reduction potential (ORP) of the antimicrobial composition ranges from about 300 mV to about 800 mV.

2. The antimicrobial composition of claim 1, wherein at least one of the following conditions is satisfied:
the clay comprises a transition metal compound;
the composition further comprises a transition metal compound.

3. The antimicrobial composition of claim 1, wherein at least one of the following conditions is satisfied:
the clay comprises a transition metal compound containing a transition metal selected from the group consisting of Ti, V, Mn, Fe, Co, Ni, Cu, Zn, Zr, Mo, Ru, Rh, Pd, Ag, Pt, Au and mixtures thereof;
the composition further comprises a transition metal compound containing a transition metal selected from the group consisting of Ti, V, Mn, Fe, Co, Ni, Cu, Zn, Zr, Mo, Ru, Rh, Pd, Ag, Pt, Au and mixtures thereof.

4. The antimicrobial composition of claim 1, wherein the clay is selected from the group consisting of a bentonite clay, a chlorite clay, an illite clay, a kaolinic clay, a montmorillonite clay, a rectorite clay, a smectite clay, and mixtures thereof.

5. The antimicrobial composition of claim 1, wherein the clay is a clay having a particle size distribution such that greater than about 20% by weight and less than about 60% by weight of particles of the clay have a particle size of less than 0.25 microns as measured by Sedigraph.

6. The antimicrobial composition of claim 1, wherein a d₅₀ of the aluminum compound ranges from 1 nm to 1000 nm.

7. The antimicrobial composition of claim 1, wherein the aluminum compound is selected from the group consisting of aluminum, an alloy containing aluminum, aluminum acetate [Al(C₂H₃O₂)₃], aluminum sulfate [Al₂(SO₄)₃], potassium aluminum sulfate [KAl(SO₄)₂], aluminum carbonate [Al₂(CO₃)₃], aluminum sulfite [Al₂(SO₃)₃], aluminum oxide [Al₂O₃], aluminum chlorate [Al(ClO₃)₃], aluminum sulfide [Al₂S₃], aluminum nitrate [Al(NO₃)₃], aluminum permanganate [Al(MnO₄)₃], aluminum hydrogen carbonate [Al(HCO₃)₃], aluminum phosphate [AlPO₄], aluminum oxalate [Al₂(C₂O₄)₃], aluminum hydrogen phosphate [Al₂(HPO₄)₃], aluminum thiosulfate [Al₂(S₂O₃)₃], aluminum chlorite [Al(ClO₂)₃], aluminum hydrogen sulfate [Al(HSO₄)₃], aluminum dihydrogen phosphate [Al(H₂PO₄)₃], aluminum phosphite [AlPO₃], aluminum potassium sulfate [KAl(SO₄)₂], hydrates thereof, and mixtures thereof.

8. The antimicrobial composition of claim 1, wherein a crystalline structure of the clay includes a transition metal compound.

9. An antimicrobial composition comprising:
a clay; and
an aluminum compound,
wherein the clay comprises a transition element compound, and
a pH of the antimicrobial composition is less than or equal to 5; and an oxidation-reduction potential (ORP) of the antimicrobial composition ranges from about 300 mV to about 800 mV.

10-11. (canceled)

12. An antimicrobial composition comprising:
a clay; and
an aluminum compound, and
a transition metal compound,
wherein a pH of the antimicrobial composition is less than or equal to 5; and an oxidation-reduction potential (ORP) of the antimicrobial composition ranges from about 300 mV to about 800 mV.

13. The antimicrobial composition of claim 12, wherein at least one of the following conditions is satisfied:
the antimicrobial composition comprises at least two transition metal compounds;
the antimicrobial composition comprises at least two aluminum compounds.

14-17. (canceled)

18. The antimicrobial composition of claim 12, wherein the transition metal compound comprises an iron compound, a copper compound, or a mixture thereof.

19. The antimicrobial composition of claim 12, wherein:
the clay is a kaolinic clay;
the transition metal compound comprises at least one selected from the group consisting of an iron (II) sulfate, an iron (III) sulfate, a copper (I) sulfate, and a copper (II) sulfate; and
the aluminum compound comprises an aluminum sulfate, a potassium aluminum sulfate, or a mixture thereof.

20. The antimicrobial composition of claim 1, wherein the pH of the antimicrobial composition ranges from 2 to 5.

21. The antimicrobial composition of claim 1, wherein a proportion of the aluminum compound ranges from 0.10 wt % to 5.0 wt %, relative to a total weight of the clay.

22. The antimicrobial composition of claim 12, wherein:
a proportion of the transition metal compound ranges from 0.10 wt % to 5.0 wt %, relative to a total weight of the clay; and
a proportion of the aluminum compound ranges from 0.10 wt % to 5.0 wt %, relative to the total weight of the clay.

23. The antimicrobial composition of claim 12, wherein a gram weight ratio of the transition metal compound to the aluminum compound ranges from 0.01:99.99 to 99.99:0.01.

24. (canceled)

25. The antimicrobial composition of claim 1, further comprising an aqueous liquid.

26. (canceled)

27. The antimicrobial composition of claim 1, further comprising at least one selected from the group consisting of a transition metal compound, a reducing agent, an antioxidant, and oxygen scavenger, a filler, a dispersant, an organic polymer, a pigment, a therapeutic agent and an antiseptic.

28. The antimicrobial composition of claim 1, wherein the clay comprises:
Fe₂O₃: 0.5-5.0 wt %;
MgO: 0.0-1.0 wt %;
Al₂O₃: 10.0-50.0 wt %;
SiO₂: 10.0-50.0 wt %;
TiO₂: 1.0-5.0 wt %;
CaO: 0.1-1.0 wt %;
Na₂O: 0.1-2.0 wt %;
K₂O: 0.1-1.0 wt %;
P₂O₅: 0.05-1.0 wt %;
Horiba S: 0.0-5.0 wt %; and
FeS₂: 0.01-7.0 wt %,
relative to a total weight of the clay.

29-45. (canceled)
